(19) United States
(12) Patent Application Publication
(10) Pub. No.: US 2012/0091885 A1
(43) Pub. Date: Apr. 19, 2012

(54) NOVEL ORGANIC ELECTROLUMINESCENT COMPOUNDS AND ORGANIC ELECTROLUMINESCENT DEVICE USING THE SAME

(75) Inventors: Young Gil Kim, Gyeonggi-do (KR); Young Jun Chop, Seoul (KR); Hyuck Joo Kwon, Seoul (KR); Bong Ok Kim, Seoul (KR); Sung Min Kim, Seoul-city (KR); Seung Soo Yoon, Seoul (KR)

(73) Assignee: Rohm and Haas Electronic Materials Korea Let., Chungcheon-do (KR)

(21) Appl. No.: 13/262,436

(22) PCT Filed: Mar. 29, 2010
(86) PCT No.: PCT/KR2010/001900

§ 371 Date: Dec. 27, 2011

(30) Foreign Application Priority Data
Mar. 31, 2009 (KR) 10-2009-0027448

(51) Int. Cl. H01L 51/54; C07D 251/20; C07C 43/21; C07C 13/66; C07C 255/50; C07C 25/22; C07D 213/16; C07D 223/22

(52) U.S. Cl. 313/504; 546/285; 544/180; 540/588; 585/27; 558/411; 570/129; 568/632

(57) ABSTRACT

Disclosed are novel organic electroluminescent compounds and organic electroluminescent devices comprising the same.

With good luminous efficiency and excellent life property, the disclosed organic electroluminescent compounds can be used to manufacture OLED devices having very good operation life.

### FIELD OF THE INVENTION

The present invention relates to novel organic electroluminescent compounds, specifically, those represented by Chemical Formula (1), and organic electroluminescent devices comprising the same.

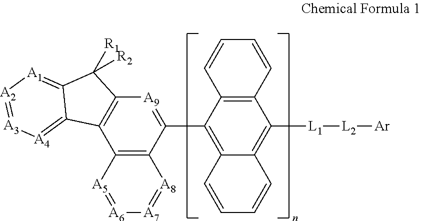

### BACKGROUND OF THE INVENTION

Among display devices, electroluminescence (EL) devices, being self-luminous type display devices, have advantages of wide visual angle, excellent contrast as well as rapid response rate. Eastman Kodak firstly developed an organic EL device employing low molecular aromatic diamine and aluminum complex as a substance for forming an electroluminescent layer, in 1987 [Appl. Phys. Lett. 51, 913, 1987].

An organic EL device is a device wherein, when charge is applied to an organic film formed between an electron injection electrode (cathode) and a hole injection electrode (anode), an electron and a hole form a pair and then become extinct with emitting light. A device can be formed on a transparent flexible substrate such as plastics. The device can be operated at a lower voltage (not more than 10 V) with relatively lower power consumption but excellent color purity, as compared to a plasma display panel or an inorganic EL display. Since the organic electroluminescent (EL) devices can develop three colors (green, blue and red), they have been focused for full colored display devices for next generation.

The most important factor to determine luminous efficiency, lifetime or the like in an organic EL device is electroluminescent material. Several properties required for such electroluminescent materials include that the material should have high luminescent quantum yield in solid state and high mobility of electrons and holes, is not easily decomposed during vapor-deposition in vacuum, and forms uniform and stable thin film.

An organic EL device is composed of anode/HIL/HTL/EML/ETL/EIL/cathode. The color of the light emitted (blue, green, red) from the organic electroluminescent device can be realized depending on how the electroluminescent layer (EML) is formed.

Electroluminescent materials are classified into host materials and dopant materials from the aspect of their functions. It is generally known that a device structure having the most excellent EL properties can be fabricated with an EL layer prepared by doping a dopant to a host. Recently, development of organic EL devices with high efficiency and long life comes to the fore as an urgent subject, and particularly urgent is development of a material with far better EL properties as compared to conventional EL materials as considering EL properties required for a medium to large sized OLED panel.

In the meanwhile, as to conventional blue materials, a number of materials have been developed and commercialized since the development of diphenylvinyl-biphenyl (DPVBi) (Compound a) by Idemitsu-Kosan. In addition to the blue material system from Idemitsu-Kosan, dinaphthylanthracene (DNA, Compound b) from Kodak, tetra(t-butyl)perylene (Compound c) system or the like have been known. However, extensive research and development should be performed with respect to these materials.

The distryl compound system of Idemitsu-Kosan, which is known to have highest efficiency up to now, has 6 lm/W of power efficiency and beneficial device lifetime of more than 30,000 hr. However, the device would have the life of only several thousand hours owing to impaired color purity by the lapse of operation time, when it is applied to a full-colored display. In case of blue electroluminescentce, it becomes advantageous from the aspect of the luminous efficiency, if the electroluminescent wavelength is shifted a little toward longer wavelength. However, it is not easy to apply the material to a display of high quality because of unsatisfactory color purity in blue. In addition, the research and development of such materials are urgently demanded because of the problems in color purity, efficiency and thermal stability.

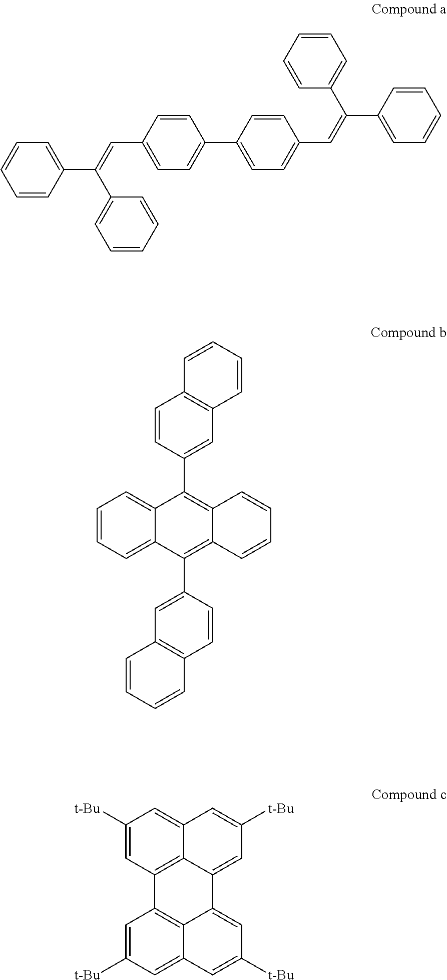

As described above, conventional materials are constituted by a single layer, not forming host-dopant thin layer, and is difficult to be used practically from the aspect of color purity and efficiency. It lacks reliable data with respect to its long life.

### Technical Problem

The object of the invention is to overcome the problems of conventional techniques as described above, and to provide organic electroluminescent compounds comprising an excellent backbone to obtain better luminous efficiency, device life and appropriate color coordinate, as compared to conventional host materials.

Another object of the invention is to provide an organic electroluminescent device of high efficiency and long life by employing the organic electroluminescent compound as electroluminescent material.

### Technical Solution

The present invention relates to organic electroluminescent compounds represented by Chemical Formula (1), and organic electroluminescent devices comprising the same. The organic electroluminescent compounds according to the invention exhibit high luminous efficiency and excellent life properties of the material, so that OLED's with very excellent operation life can be manufactured therefrom.

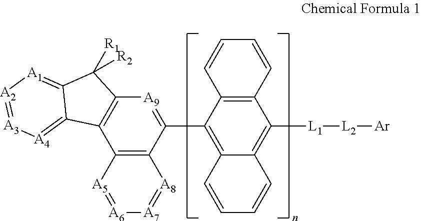

wherein

A₁ through A₉ independently represent CR₃₁ or N;

L₁ and L₂ independently represent a chemical bond,
(C6-C30)arylene with or without substituent(s),
(C3-C30)heteroarylene with or without substituent(s), 5- to 7-membered heterocycloalkylene with or without substituent(s), substituted or unsubstituted 5- to 7-membered heterocycloalkylene fused with one or more aromatic ring (s), (C3-C30) cycloalkylene with or without substituent(s), substituted or unsubstituted
(C3-C30)cycloalkylene fused with one or more aromatic ring(s), adamantylene with or without substituent(s),
(C7-C30)bicycloalkylene with or without substituent(s),
(C2-C30)alkenylene with or without substituent(s),
(C2-C30)alkynylene with or without substituent(s),
(C6-C30)ar(C1-C30)alkylene with or without substituent(s),
(C1-C30)alkylenethio with or without substituent(s),
(C1-C30)alkyleneoxy with or without substituent(s),
(C6-C30)aryleneoxy with or without substituent(s),
(C6-C30)arylenethio with or without substituent(s), —O— or —S—;

R₁, R₂, R₃₁ and Ar independently represent hydrogen, deuterium, halogen, (C1-C30)alkyl with or without substituent(s), (C6-C30) aryl with or without substituent(s), substituted or unsubstituted
(C6-C30)aryl fused with one or more (C3-C30)cycloalkyl(s) with or without substituent(s), (C3-C30)heteroaryl with or without substituent(s), 5- to 7-membered heterocycloalkyl with or without substituent(s), substituted or unsubstituted 5- to 7-membered heterocycloalkyl fused with one or more aromatic ring(s),
(C3-C30)cycloalkyl with or without substituent(s), substituted or unsubstituted (C3-C30)cycloalkyl fused with one or more aromatic ring(s), adamantyl with or without substituent(s),
(C7-C30) bicycloalkyl with or without substituent (s), cyano, NR₁₁R₁₂, BR₁₃R₁₄, PR₁₅R₁₆, P(═O)R₁₇R₁₈ [wherein R₁₁ through R₁₈ independently represent (C1-C30)alkyl with or without substituent(s),
(C6-C30)aryl with or without substituent(s) or (C3-C30)heteroaryl with or without substituent(s)], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s),
(C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s),
(C2-C30)alkynyl with or without substituent(s),
(C6-C30)aryloxycarbonyl with or without substituent(s),
(C1-C30)alkoxycarbonyloxy with or without substituent(s),
(C1-C30)alkylcarbonyloxy with or without substituent(s),
(C6-C30)arylcarbonyloxy with or without substituent(s),
(C6-C30)aryloxycarbonyloxy with or without substituent(s), carbonxyl, nitro,

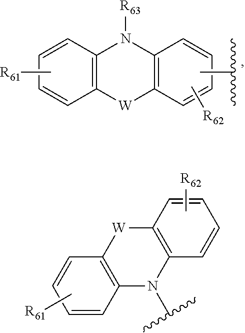

or hydroxyl, or each of them may be linked to an adjacent substituent via (C3-C30) alkylene or (C3-C30) alkenylene with or without a fused ring to form an alicyclic ring or a mono- or polycyclic aromatic ring;

W represents —(CR₅₁R₅₂)ₙ—, —(R₅₁) C═C (R₅₂)—, —N(R₅₃)—, —S—, —O—, —Si(R₅₄) (R₅₅)—, —P(R₅₆)—, —P(═O) (R₅₇)—, —C(═O)— or —B(R₅₈)—, and R₅₁ through R₅₈ and R₆₁ through R₆₃ are defined as for R₁ and R₂;

each of the heterocycloalkyl and heteroaryl contains one or more heteroatom(s) selected from B, N, O, S, P(═O), Si and P;

m represents an integer 1 or 2; and

n represents an integer 1 or 2.

The ‘alkyl’, ‘alkoxy’ and other substituents containing ‘alkyl’ moiety described herein include both linear and branched species.

The term ‘aryl’ described herein represents an organic radical derived from aromatic hydrocarbon by deleting one hydrogen atom therefrom. An aryl group may be a monocyclic and fused ring system, each ring of which suitably contains from 4 to 7, preferably from 5 or 6 cyclic atoms. Structures wherein two or more aryl groups are combined through chemical bond (s) are also included. Specific examples include phenyl, naphthyl, biphenyl, anthryl, indenyl, fluorenyl, phenanthryl, triphenylenyl, pyrenyl, perylenyl, chrysenyl, naphtacenyl, fluoranthenyl and the like, but are not restricted thereto. The naphthyl may be 1-naphthyl or 2-naphthyl, the anthryl may be 1-anthryl, 2-anthryl or 9-anthryl, and the fluorenyl may be any one of 1-fluorenyl, 2-fluorenyl, 3-fluorenyl, 4-fluorenyl and 9-fluorenyl.

The term ‘heteroaryl’ described herein means an aryl group containing from 1 to 4 heteroatom (s) selected from B, N, O, S, P (═O), Si and P for the aromatic cyclic backbone atoms, and carbon atom(s) for remaining aromatic cyclic backbone atoms. The heteroaryl may be a 5- or 6-memberedmonocyclic heteroaryl or a polycyclic heteroaryl which is fused with one or more benzene ring (s), and may be partially saturated. The structures having one or more heteroaryl group(s) bonded through a single bond are also included. The heteroaryl groups may include divalent aryl groups of which the heteroatoms are oxidized or quarternized to form N-oxides, quaternary salts, or the like. Specific examples include monocyclic heteroaryl groups such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups such as benzofuranyl, benzothiophenyl, isobenzofuranyl, benzimidazolyl, benzothiazolyl, benzisothiazolyl, benzisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenanthridinyl and benzodioxolyl; and corresponding N-oxides (for example, pyridyl N-oxide, quinolyl N-oxide) and quaternary salts thereof; but they are not restricted thereto.

The alkyl groups in ‘(C1-C30)alkyl, tri(C1-C30)alkylsilyl, di(C1-C30)alkyl(C6-C30)arylsilyl, (C6-C30)ar(C1-C30)alkyl, (C1-C30)alkyloxy, (C1-C30)alkylthio, (C1-C30)alkyloxycarbonyl, (C1-C30)alkylcarbonyl, (C1-C30)alkyloxycarbonyloxy, (C1-C30)alkylcarbonyloxy’ described in the present specification may have restricted carbon number from 1 to 20, or from 1 to 10. The aryl groups in ‘ (C6-C30) aryl, di (C1-C30) alkyl (C6-C30) arylsilyl, tri(C6-C30)arylsilyl, (C6-C30)ar(C1-C30)alkyl, (C6-C30)aryloxy, (C6-C30)arylthio, (C6-C30)arylcarbonyl, (C6-C30)aryloxycarbonyl, (C6-C30)arylcarbonyloxy or (C6-C30)aryloxycarbonyloxy’ may have restricted carbon number from 6 to 20, or from 6 to 12. The heteroaryl groups in ‘(C3-C30)heteroaryl’ may have restricted carbon number from 4 to 20, or from 4 to 12. The cycloalkyl groups in ‘(C3-C30)cycloalkyl’ may have restricted carbon number from 3 to 20, or from 3 to 7. The alkenyl or alkynyl of ‘(C2-C30)alkenyl or alkynyl’ may have restricted carbon number from 2 to 20, or from 2 to 10.

The term ‘substituted or unsubstituted (or with or without) substituent(s)’ described herein means having one or more substituent(s) independently selected from deuterium, halogen, (C1-C30) alkyl with orwithout halogen substituent (s), (C6-C30) aryl, (C3-C30)heteroaryl with or without (C6-C30)aryl substituent(s), a 5- to 7-membered heterocycloalkyl containing one or more heteroatom(s) selected from B, N, O, S, P(═O), Si and P, a 5- to 7-membered heterocycloalkyl fused with one or more aromatic ring (s), (C3-C30)cycloalkyl, (C6-C30)cycloalkyl fused with one or more aromatic ring(s), tri(C1-C30)alkylsilyl, di(C1-C30)alkyl(C6-C30)arylsilyl, tri(C6-C30)arylsilyl, adamantyl, (C7-C30)bicycloalkyl, (C2-C30)alkenyl, (C2-C30)alkynyl, cyano, carbazolyl, NR₂₁R₂₂, BR₂₃R₂₄, PR₂₅R₂₆, P(═O)R₂₇R₂₈ [wherein R₂₁ through R₂₈ independently represent (C1-C30)alkyl, (C6-C30)aryl or (C3-C30)heteroaryl], (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro and hydroxyl; or that adjacent substituent(s) are linked together to form a ring.

In Chemical Formula (1), L₁ and L₂ are independently selected from a chemical bond; arylene groups such as phenylene, naphthylene, anthracenylene, biphenylene, fluorenylene, triphenylenylene, fluoranthenylene, chrysenylene, terphenylene, phenanthrylene, pyrenylene and perylenylene; and heteroarylene groups such as pyridinylene, pyrazinylene, furylene, thienylene, selenophenylene, quinolinylene, quinoxalinylene, phenanthrolinylene, group

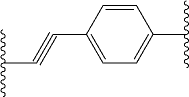

and group

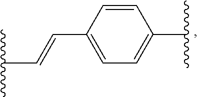

but they are not restricted thereto. Those groups may be further substituted as described for Chemical Formula (1).

Each group of R₁, R₂, R₃₁ and Ar is independently selected from aryl groups such as phenyl, naphthyl, anthryl, biphenyl, fluorenyl, phenanthryl, pyrenyl and perylenyl; heteroaryl groups such as pyridinyl, pyrazinyl, furyl, thienyl, selenophenyl, quinolinyl, quinoxalinyl, phenanthrolinyl, carbazolyl and benzopiperidinyl; aryl groups fused with cycloalkyl, such as tetrahydronaphthyl; heterocycloalkyl groups fused with one or more aromatic ring(s), such as benzopiperidino, dibenzomorpholino and dibenzoazepino; NR₇₁R₇₂, BR₇₃R₇₄, PR₇₅R₇₆, and P(═O)R₇₇R₇₈ [wherein, R₇₁ through R₇₈ independently represent substituted or unsubstituted (C1-C30) alkyl, substituted or unsubstituted (C6-C30)aryl or substituted or unsubstituted(C3-C30)heteroaryl], but not being restricted thereto, and each group may be further substituted by a substituent, as described for Chemical Formula (1).

Each group

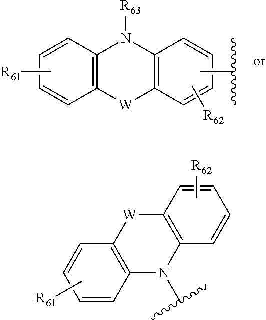

can be exemplified by the following structures.

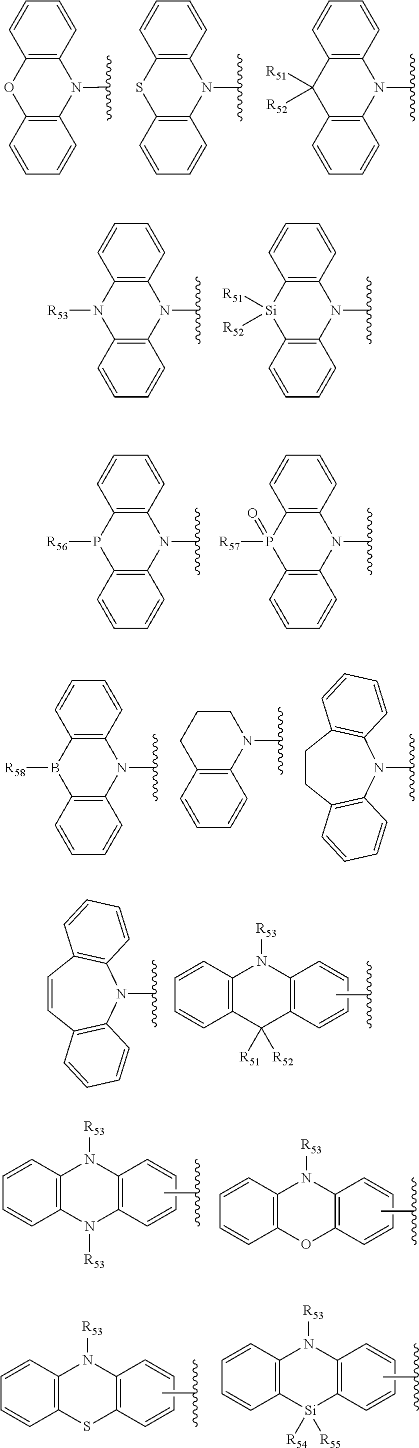

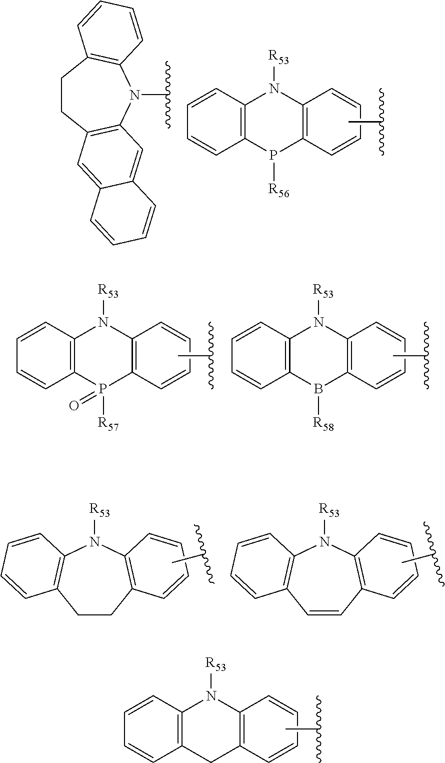

wherein, R₅₁ through R₅₈ independently represent substituted or unsubstituted (C1-C30)alkyl, substituted or unsubstituted (C6-C30)aryl, or substituted or unsubstituted (C3-C30)heteroaryl, or each of them may be linked to an adjacent substituent via (C3-C30) alkylene or (C3-C30) alkenylene with or without a fused ring to form an alicyclic ring, or a monocyclic or polycyclic aromatic ring.

The organic electroluminescent compounds represented by Chemical Formula (1) can be specifically exemplified by the following compounds, but they are not restricted thereto.

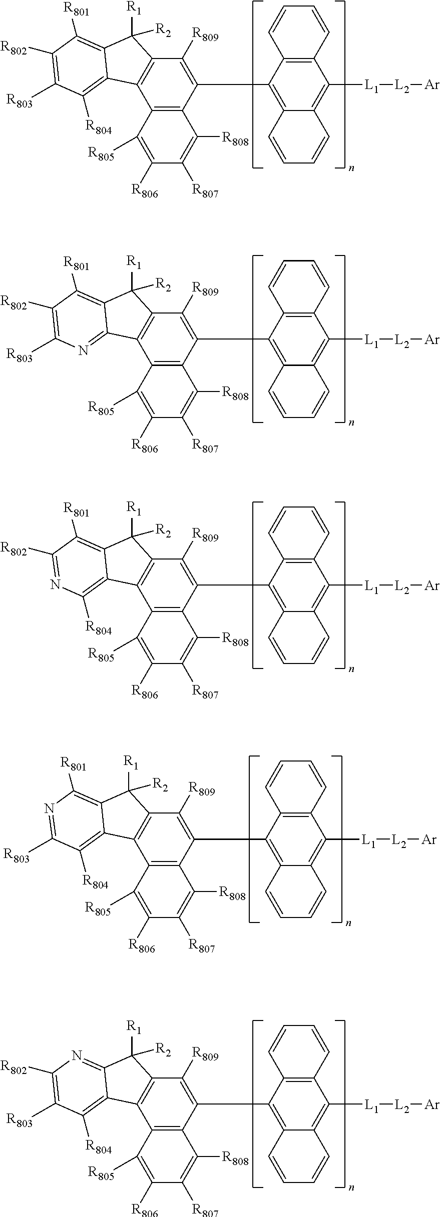

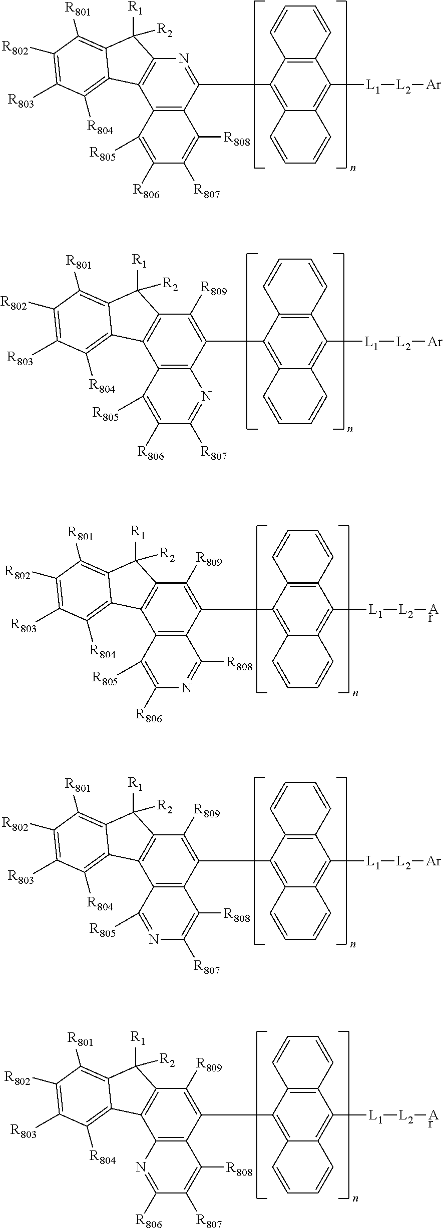

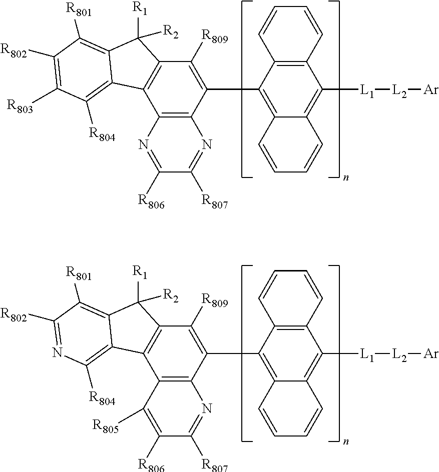

In the formulas, L₁, L₂, Ar and n are defined as in Chemical Formula (1); and R₈₀₁ and R₈₀₉ are defined as for R₁ and R₂ in Chemical Formula (1).

The organic electroluminescent compounds according to the present invention can be more specifically exemplified by the following compounds, but they are not restricted thereto.

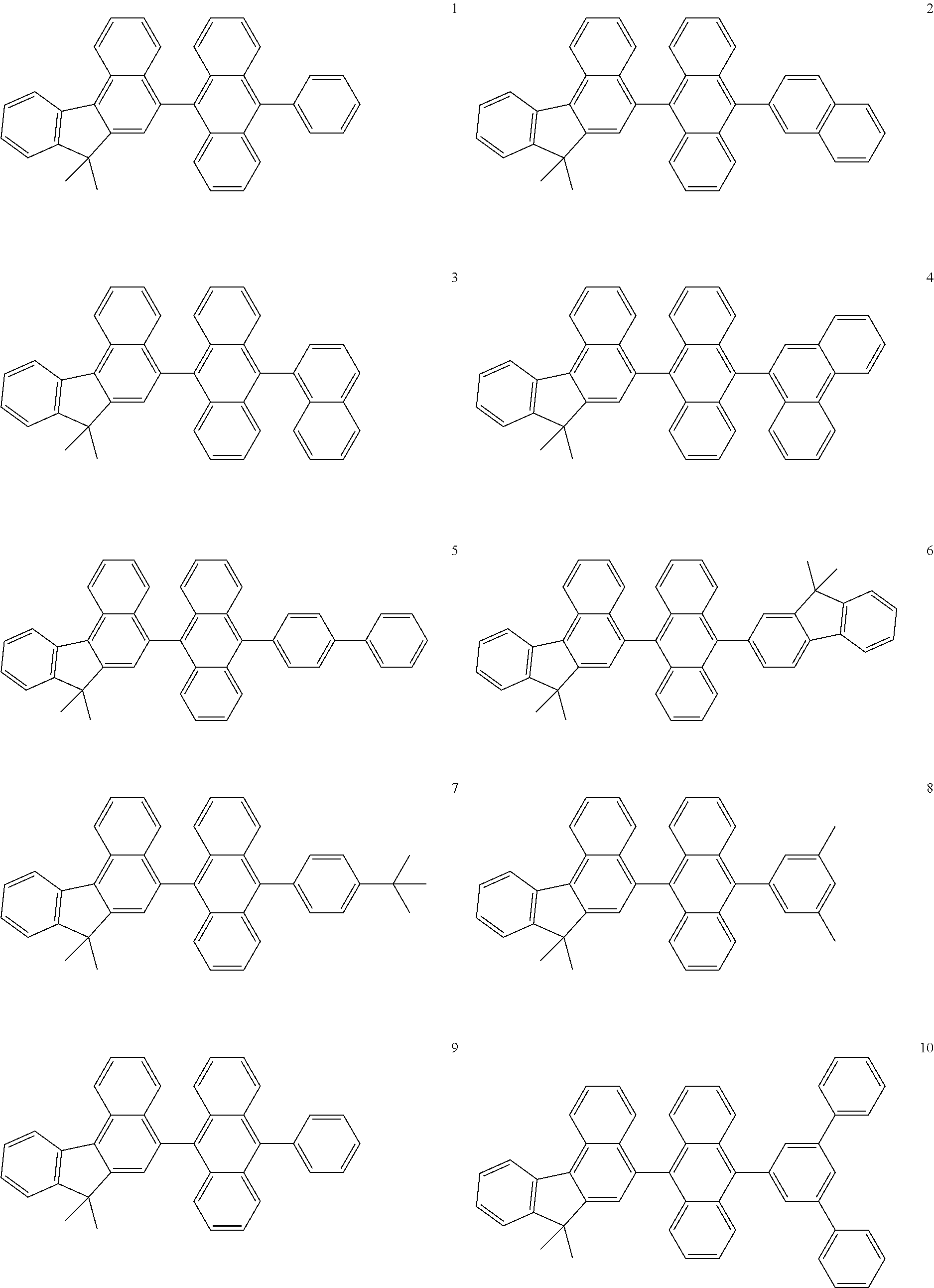

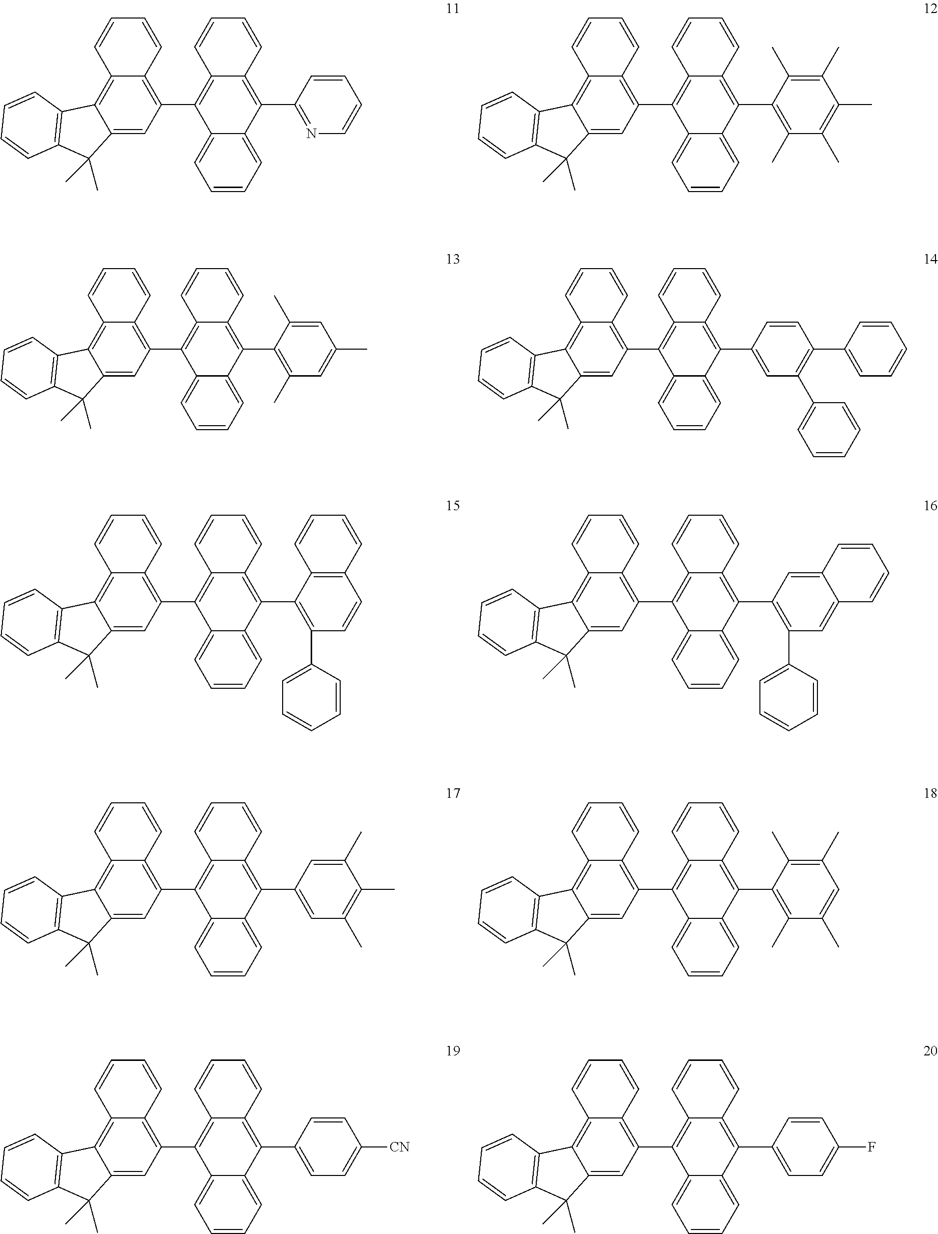

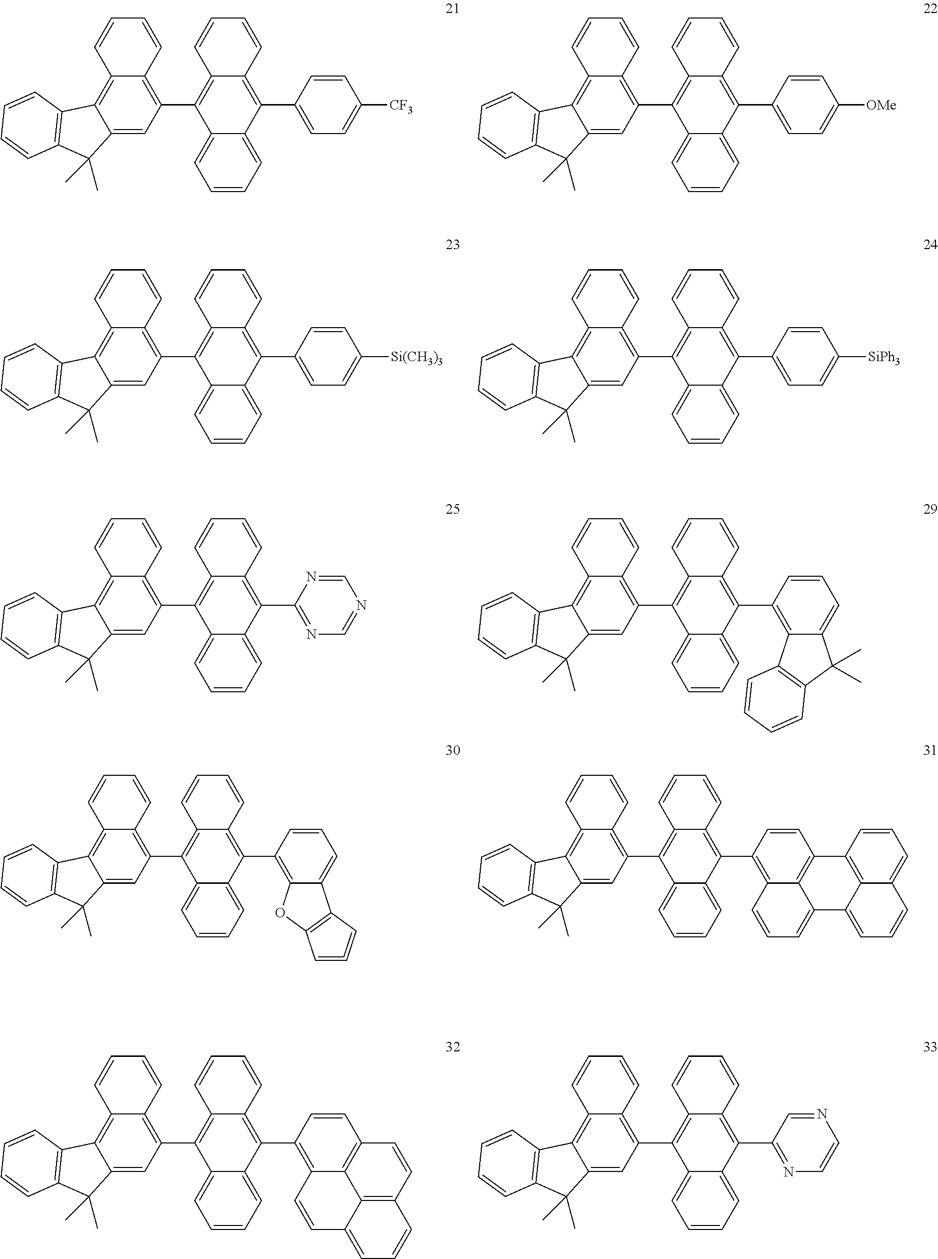

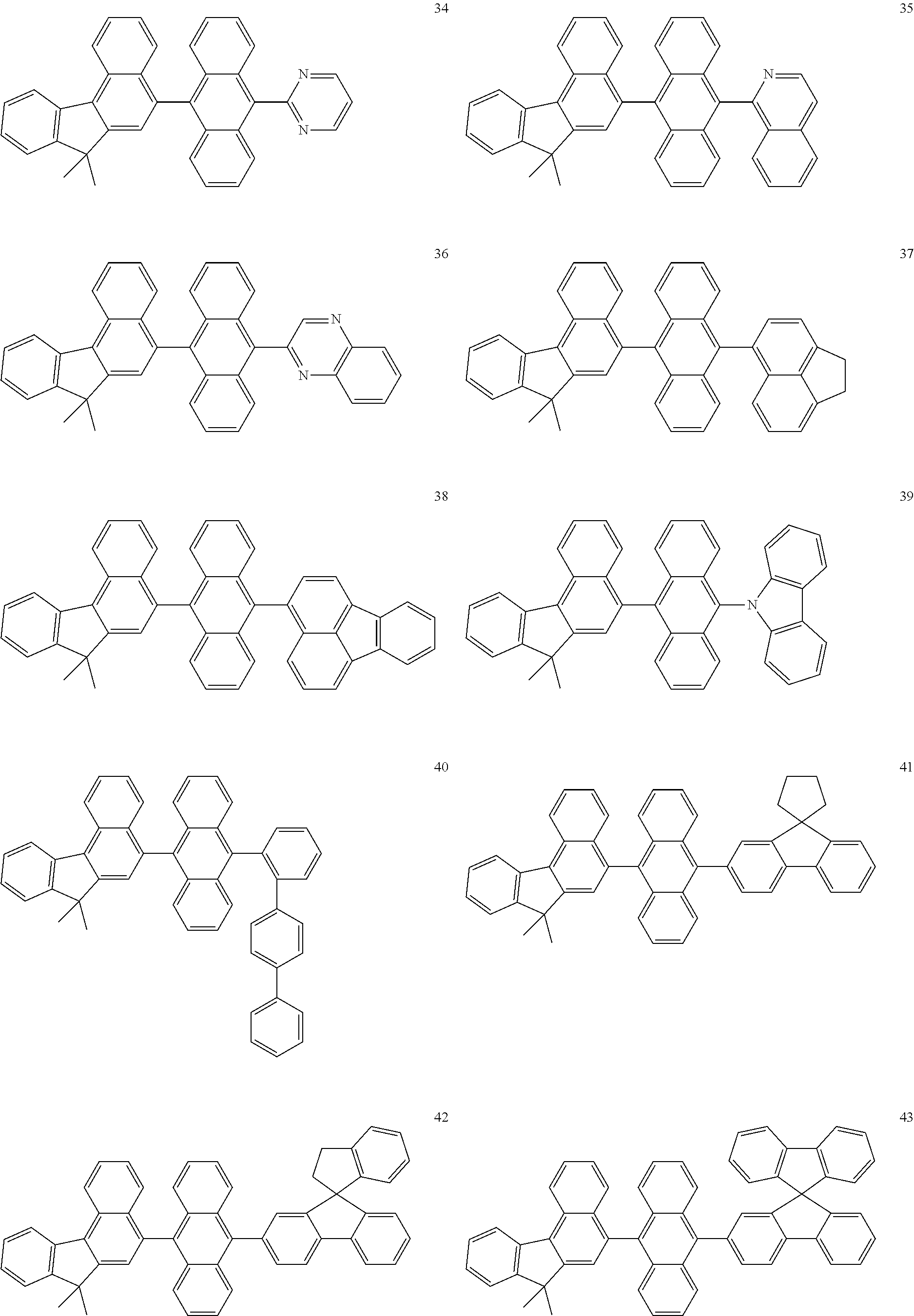

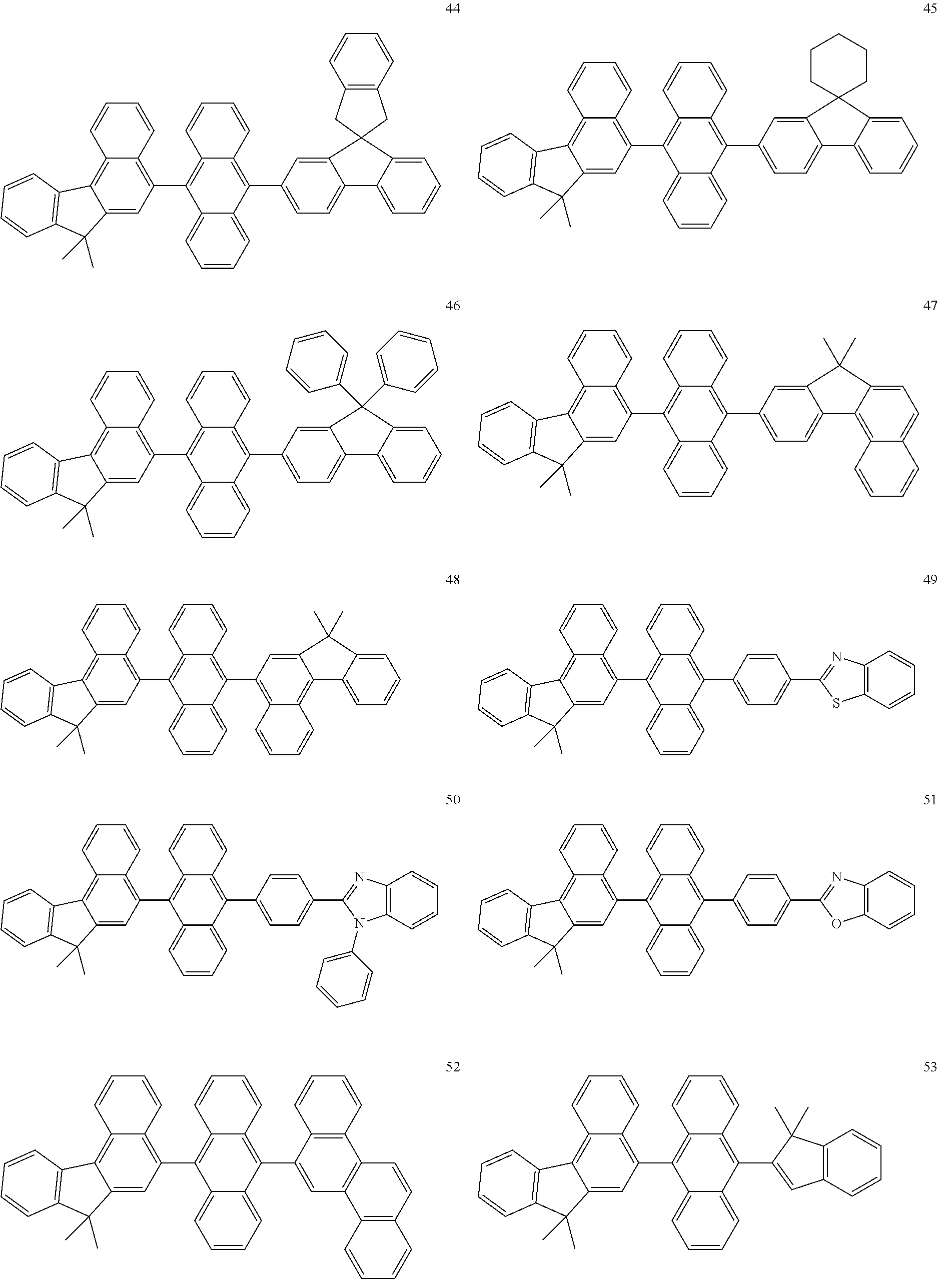

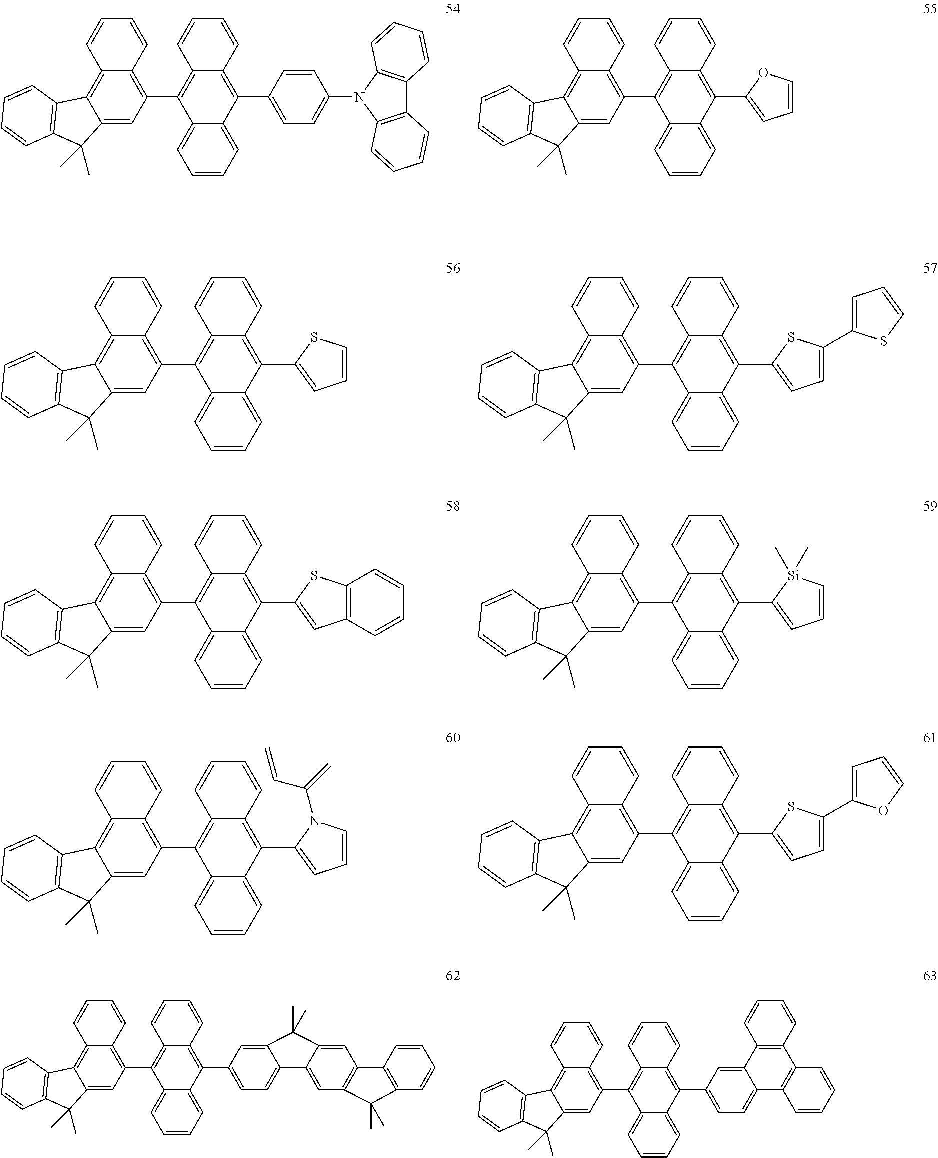

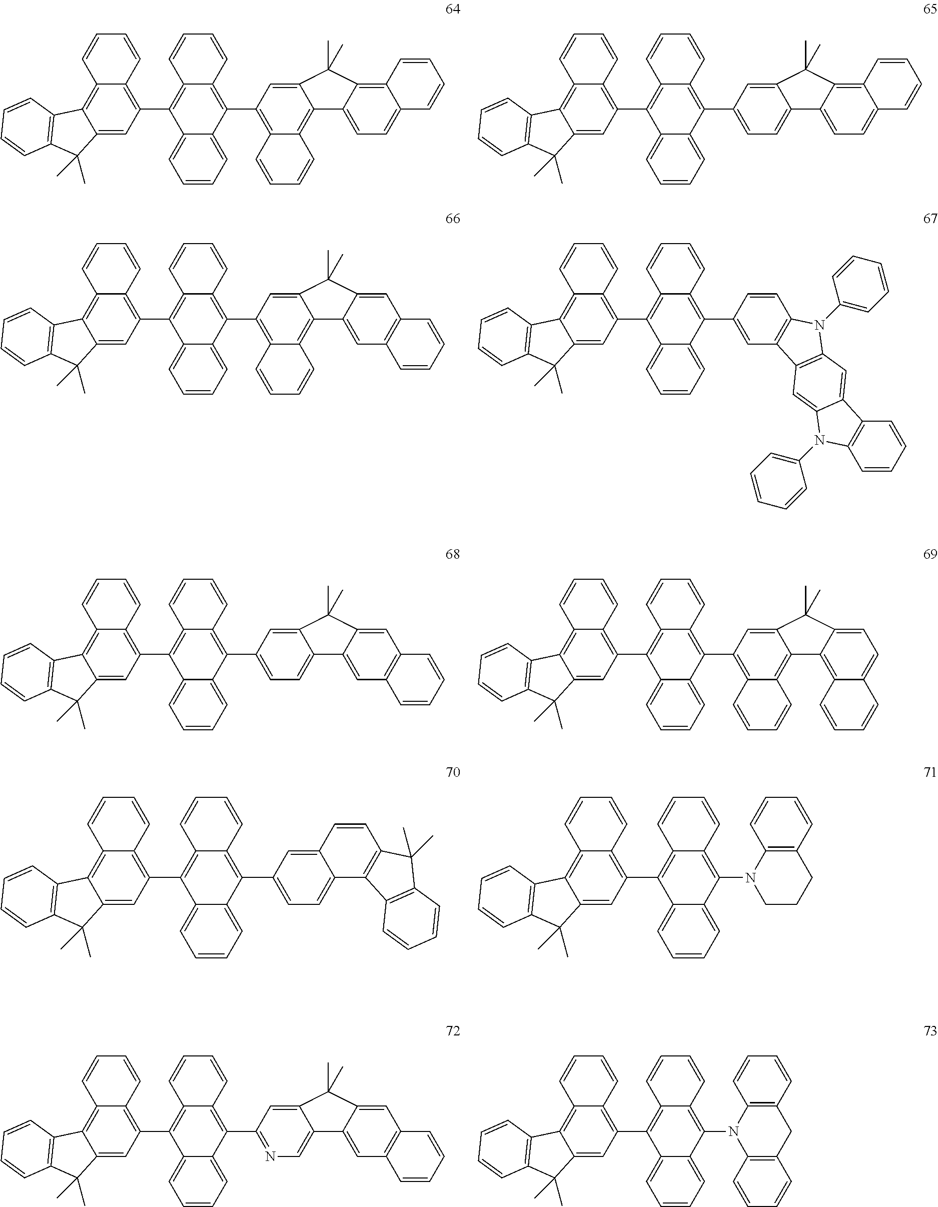

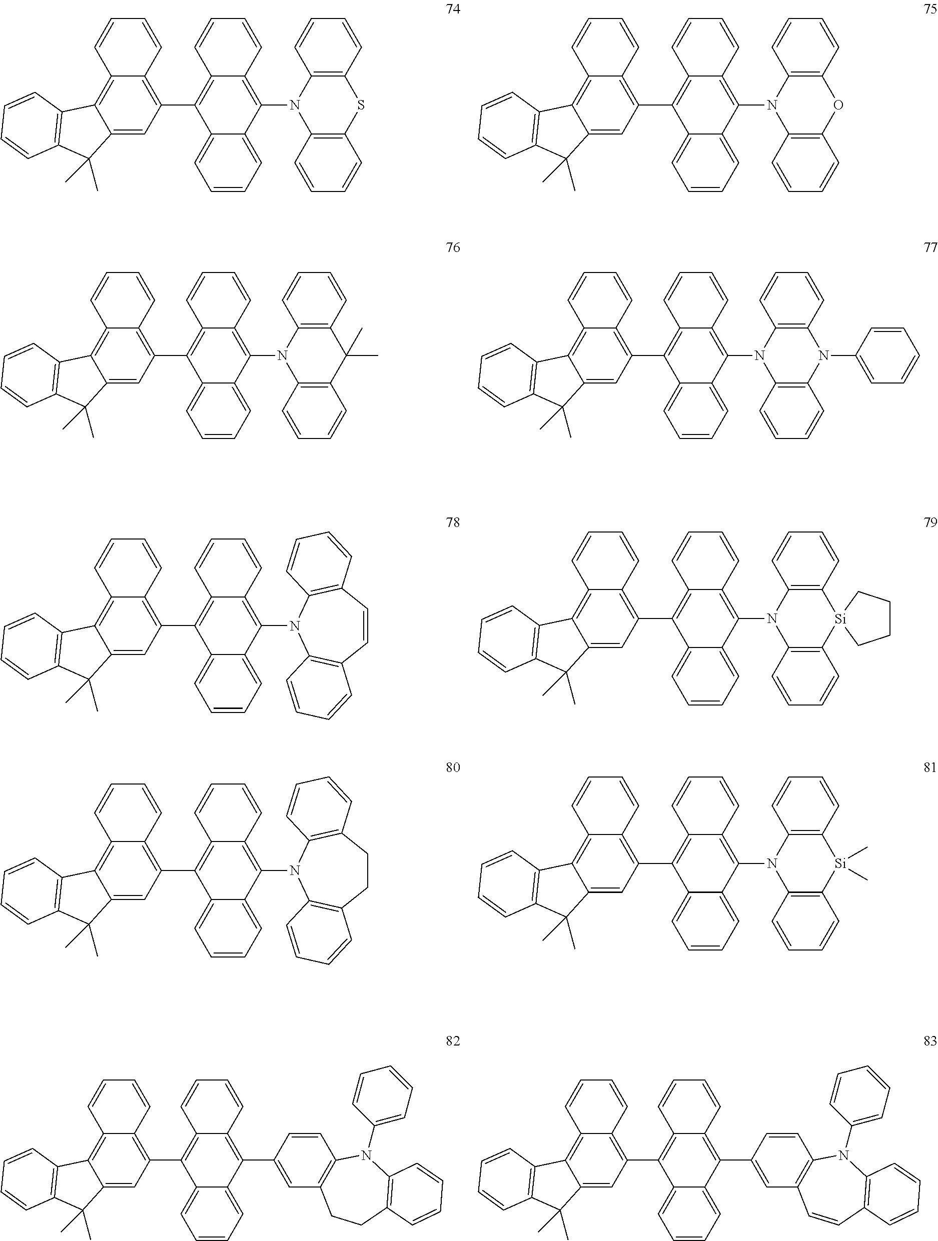

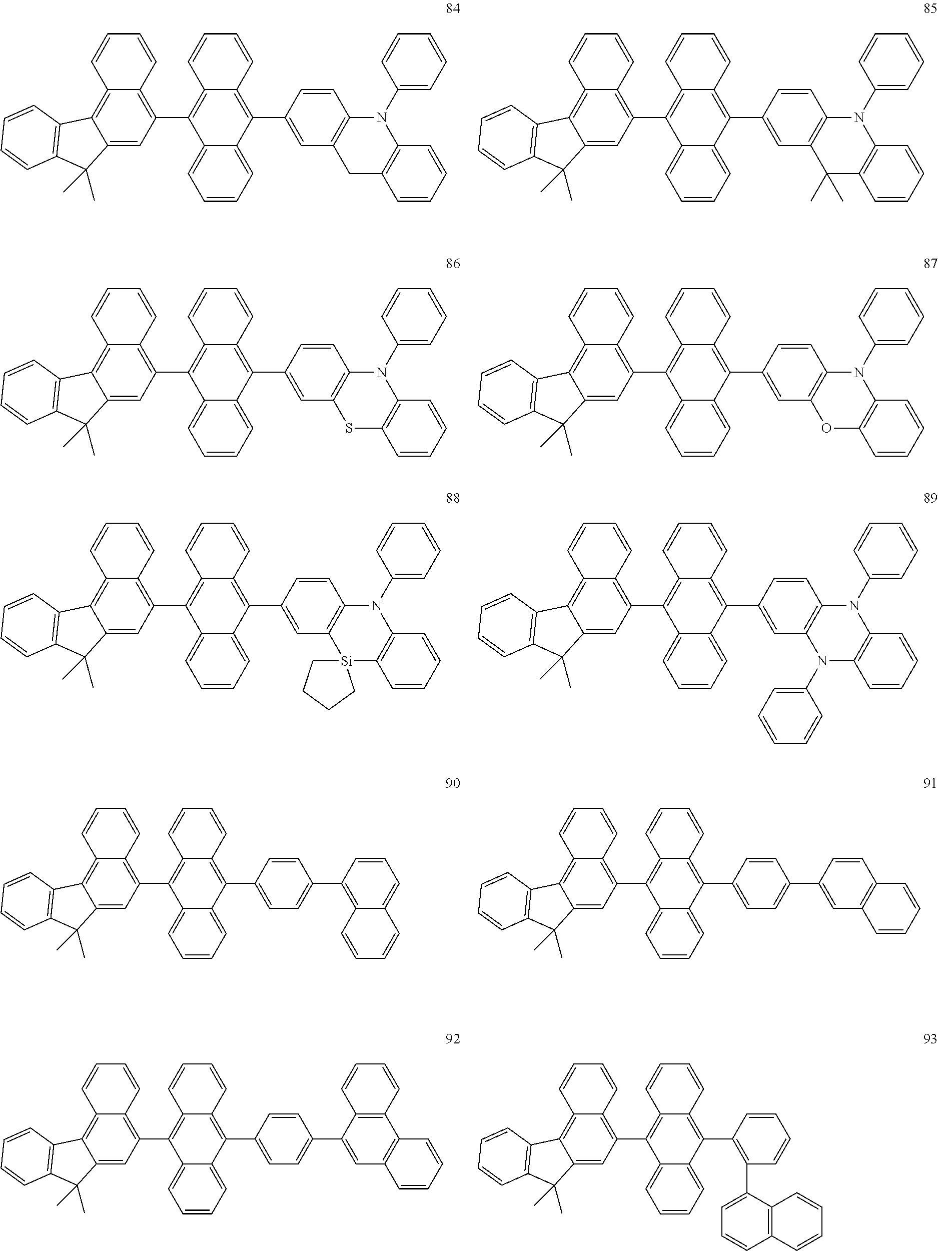

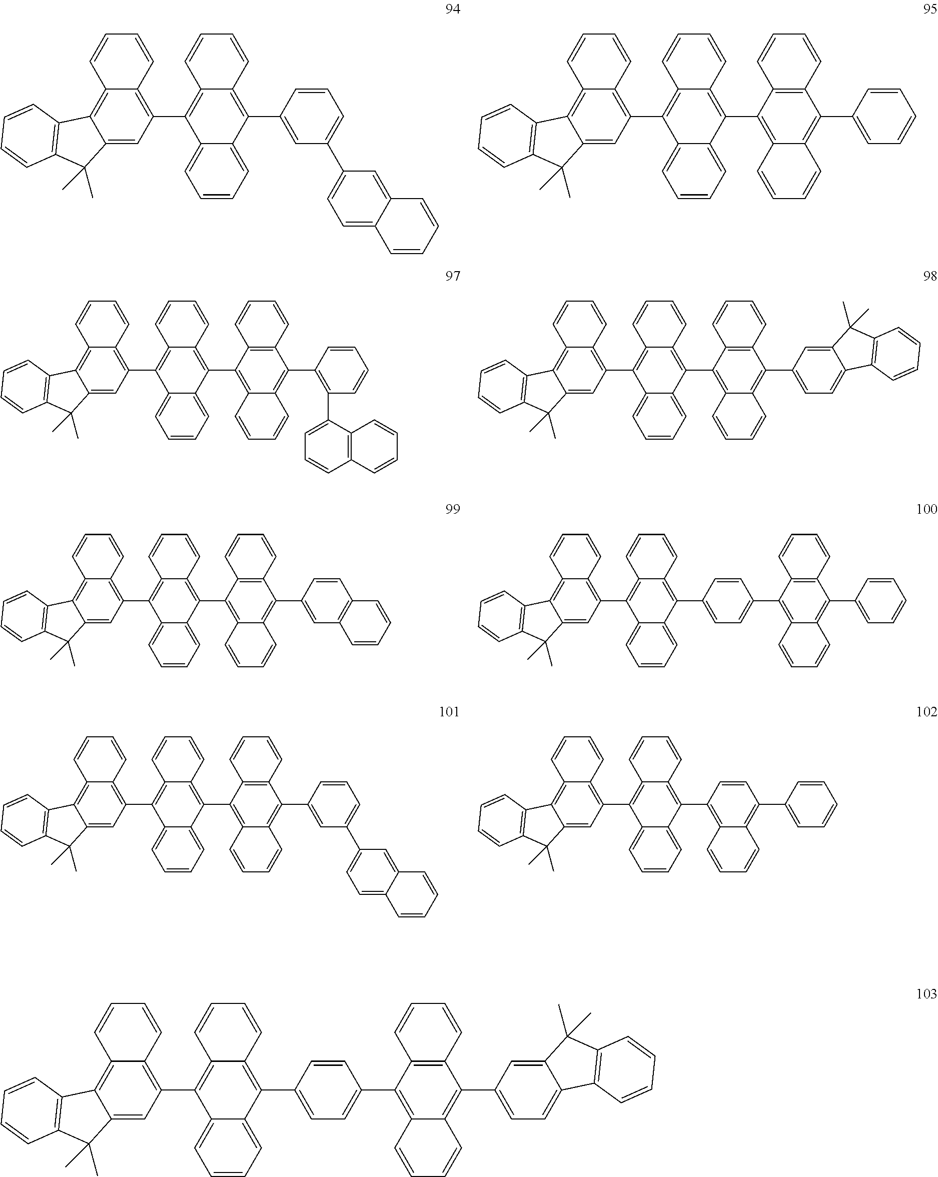

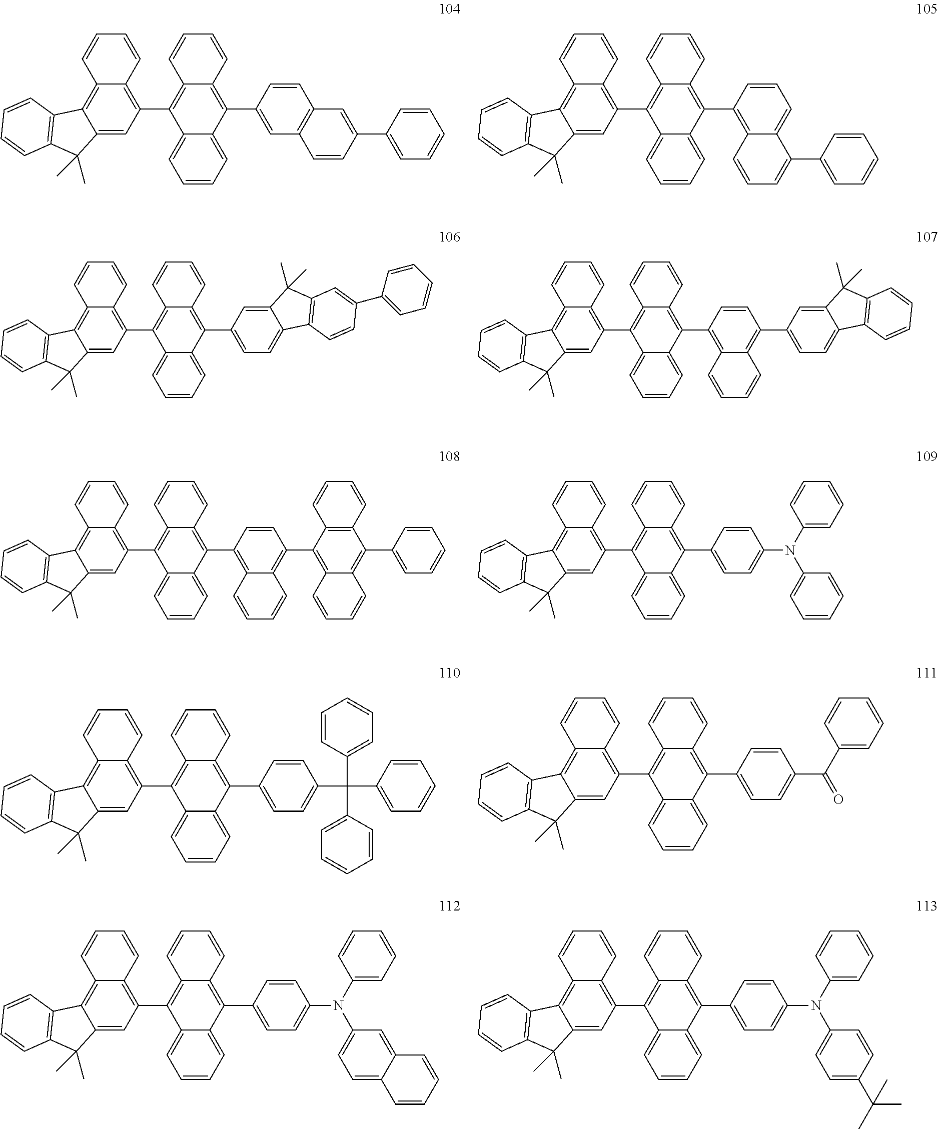

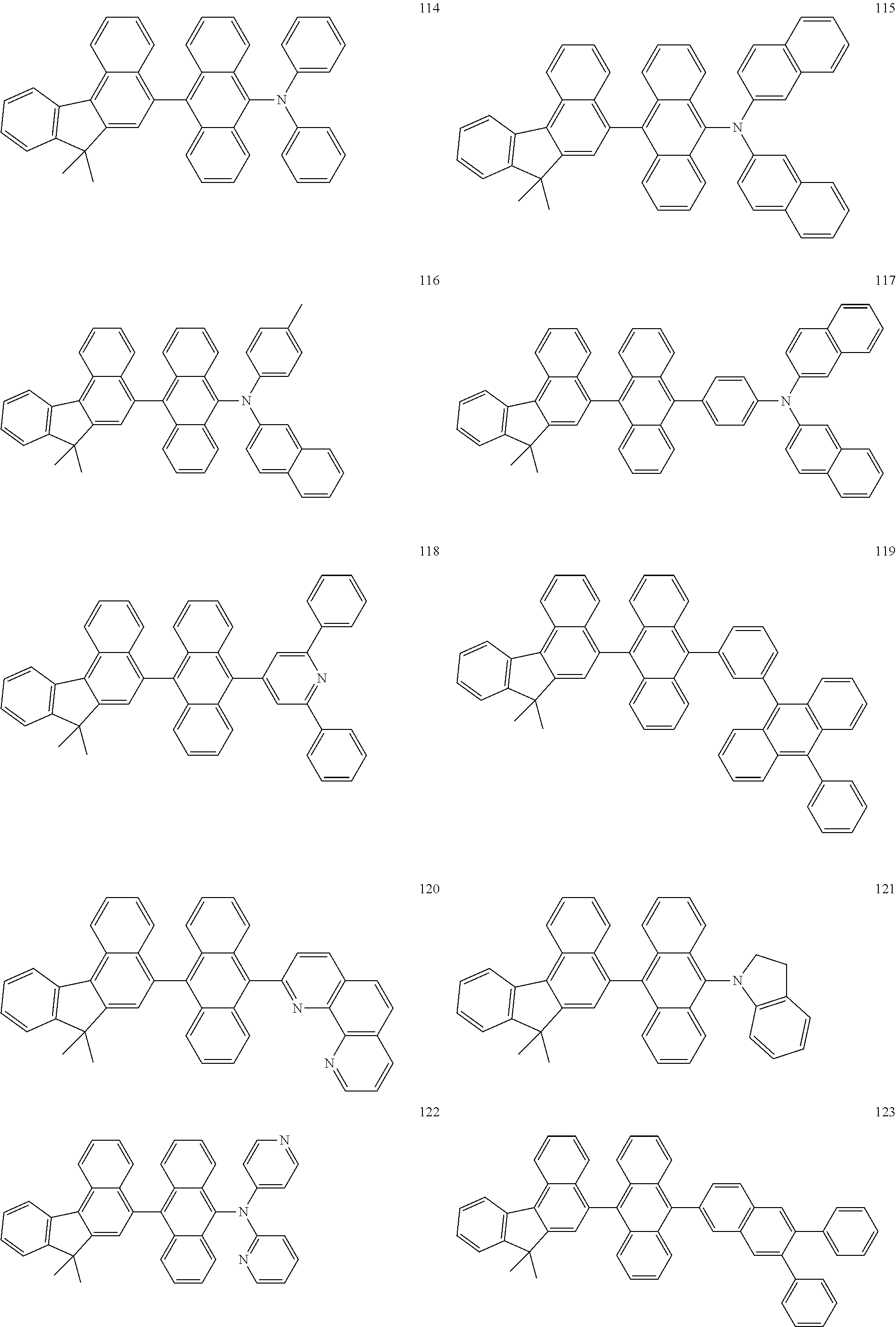

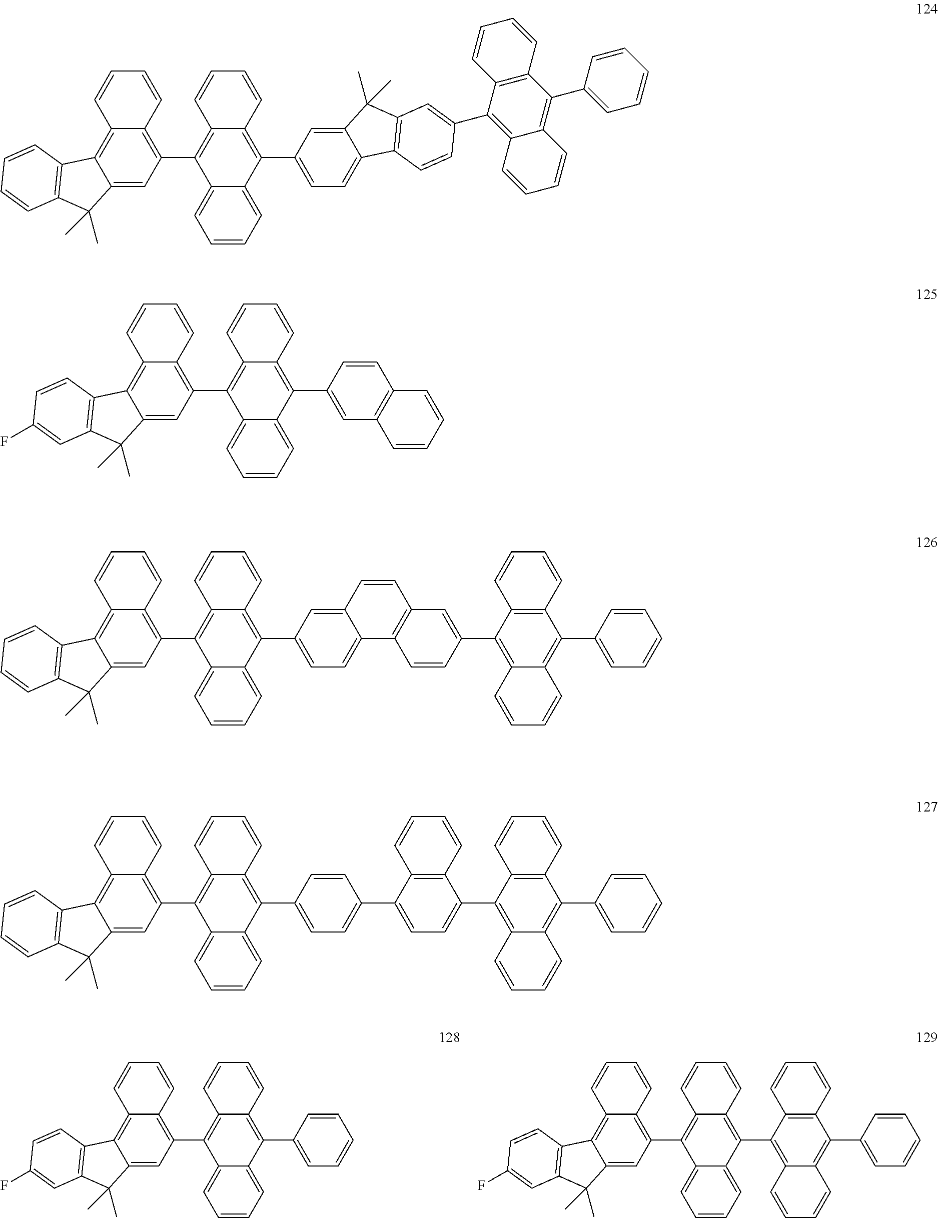

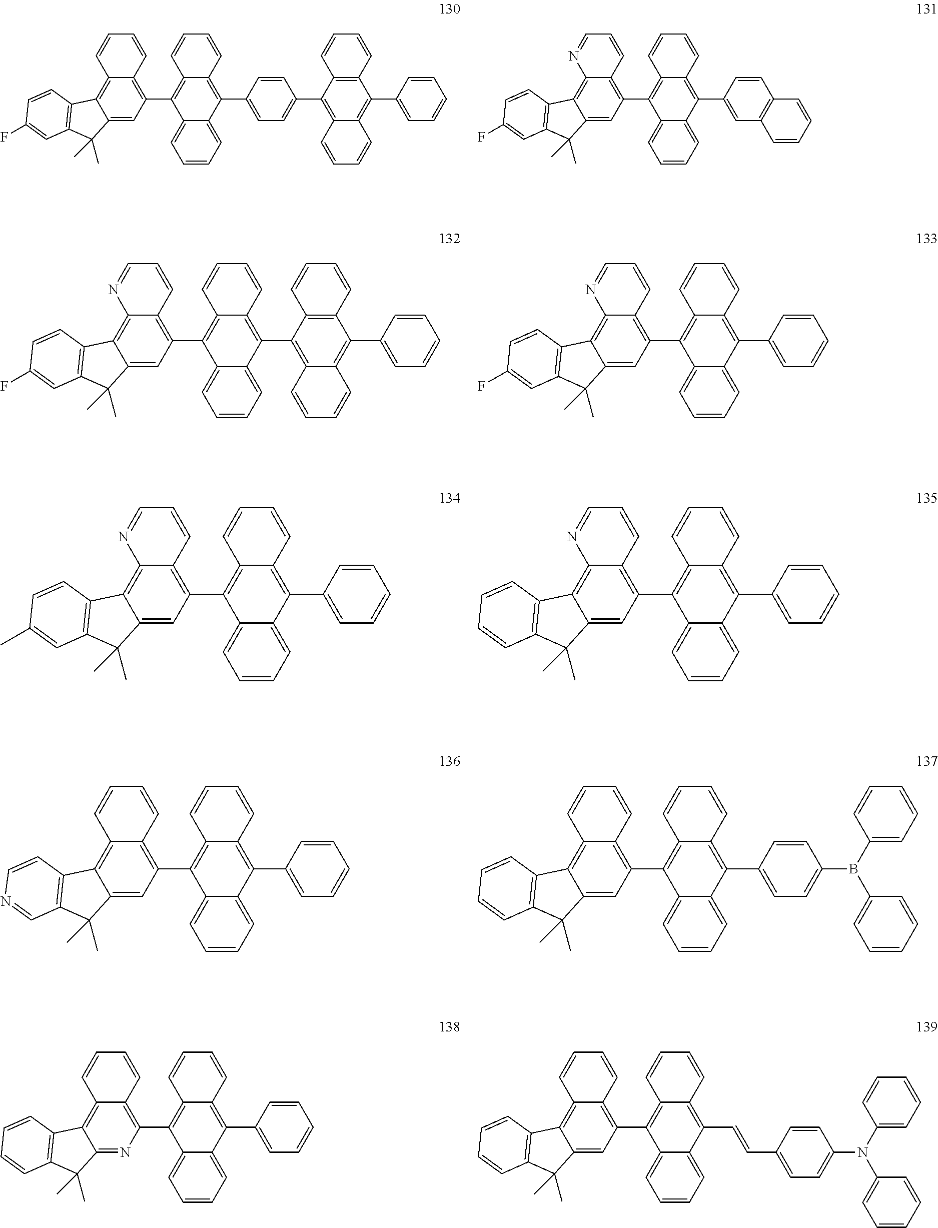

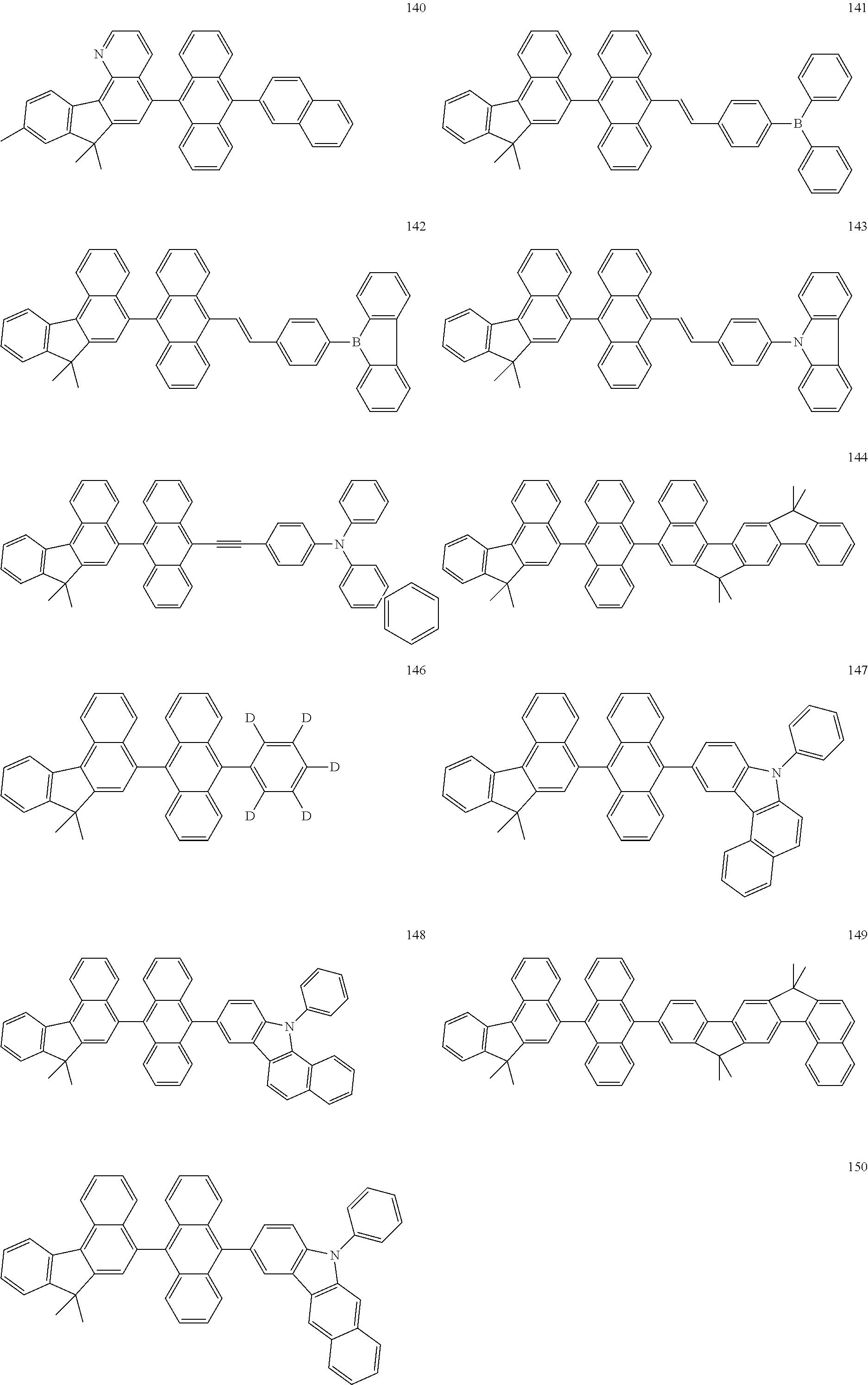

The process for preparing the organic electroluminescent compounds according to the present invention is exemplified by Reaction Scheme (1), but not being restricted thereto.

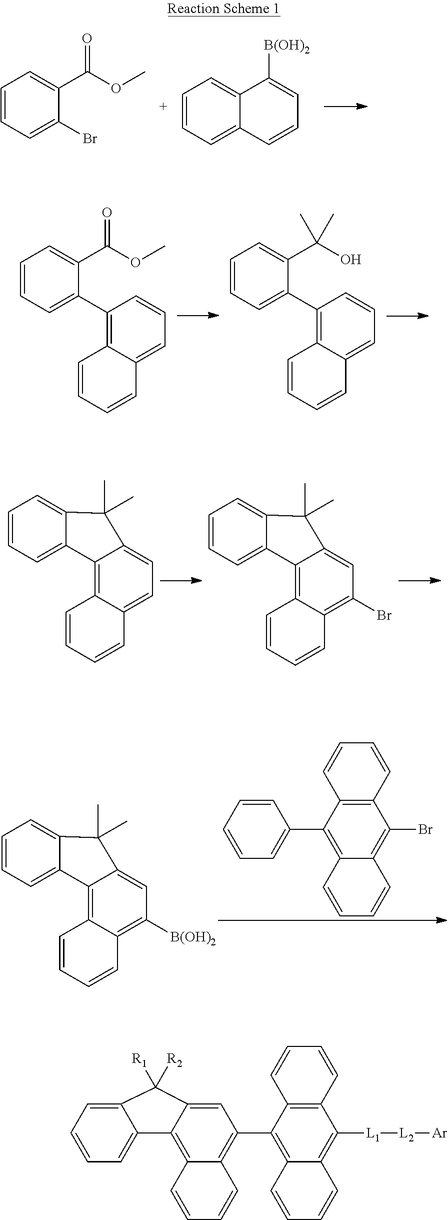

In the reaction scheme, R₁ and R₂, L₁ and L₂, and Ar are defined as in Chemical Formula (1).

The present invention also provides an organic electroluminescent device which is comprised of a first electrode; a second electrode; and at least one organic layer(s) interposed between the first electrode and the second electrode; wherein the organic layer comprises one or more organic electroluminescent compound(s) represented by Chemical Formula (1). The organic electroluminescent compound is employed as host material of the electroluminescent layer.

The organic electroluminescent device according to the present invention is characterized in that the organic layer comprises an electroluminescent layer containing one or more organic electroluminescent compound (s) represented by Chemical Formula (1), as well as one or more dopant(s). The dopant to be applied to an organic electroluminescent device according to the invention is not particularly restricted, but preferably selected from the compounds represented by Chemical Formula (2) or (3):

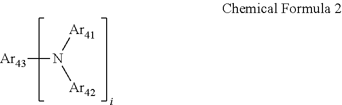

wherein,

Ar₄₁ and Ar₄₂ independently represent (C1-C30)alkyl with or without substituent(s), (C6-C30) aryl with or without substituent (s), (C4-C30) heteroaryl with or without substituent (s),
(C6-C30)arylamino with or without substituent(s),
(C1-C30)alkylamino, 5- to 7-membered heterocycloalkyl with or without substituent(s), substituted or unsubstituted 5- to 7-membered heterocycloalkyl fused with one or more aromatic ring (s),
(C3-C30)cycloalkyl with or without substituent(s), or substituted or unsubstituted (C3-C30) cycloalkyl fused with one or more aromatic ring (s), or Ar₄₂ and Ar₄₂ may be linked together via (C3-C30) alkylene or (C3-C30)alkenylene with or without a fused ring to form an alicyclic ring or a mono- or polycyclic aromatic ring;

when i is 1, Ar₄₃ represents (C6-C30)aryl with or without substituent(s), (C4-C30)heteroaryl with or without substituent(s) or a substituent selected from the following structures;

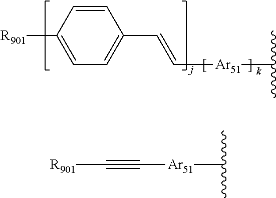

when i is 2, Ar₄₃ represents (C6-C60)arylene with or without substituent(s), (C4-C30)heteroarylene with or without substituent(s) or a substituent selected from the following structures;

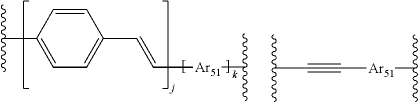

Ar₅₁ represents (C6-C60) arylene with or without substituent (s) or (C4-C30)heteroarylene with or without substituent(s);

R₉₀₁ independently represents hydrogen, deuterium, (C1-C30)alkyl with or without substituent(s) or (C6-C30)aryl with or without substituent(s);

each one of the heterocycloalkyl and heteroaryl contains one or more heteroatom(s) selected from B, N, O, S, P(═O), Si and P;

i represents an integer from 1 to 4;

j represents an integer from 1 to 4; and

k represents an integer 0 or 1:

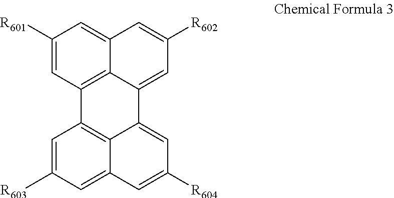

wherein,

R₆₀₁ through R₆₀₄ independently represent hydrogen, deuterium, halogen, (C1-C30)alkyl with or without substituent(s), (C6-C30) aryl with or without substituent(s), (C6-C30)heteroaryl with or without substituent(s), 5- to 7-membered heterocycloalkyl with or without substituent(s), substituted or unsubstituted 5- to 7-membered heterocycloalkyl fused with one or more aromatic ring (s),
(C3-C30)cycloalkyl with or without substituent(s), substituted or unsubstituted (C3-C30)cycloalkyl fused with one or more aromatic ring(s), adamantyl with or without substituent(s),
(C7-C30) bicycloalkyl with or without substituent (s), cyano, NR₄₁R₄₂, BR₄₃R₄₄, PR₄₅R₄₆, P(═O) R₄₇R₄₈ [wherein R₄₁ through R₄₈ independently represent (C1-C30)alkyl with or without substituent(s),
(C6-C30)aryl with or without substituent(s) or (C3-C30)heteroaryl with or without substituent(s)], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s),
(C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s),
(C2-C30)alkynyl with or without substituent(s),
(C6-C30)aryloxycarbonyl with or without substituent(s),
(C1-C30)alkoxycarbonyloxy with or without substituent(s),
(C1-C30)alkylcarbonyloxy with or without substituent(s),
(C6-C30)arylcarbonyloxy with or without substituent(s),
(C6-C30)aryloxycarbonyloxy with or without substituent(s), carboxyl, nitro or hydroxyl, or each of them may be linked to an adjacent carbon atom via (C3-C30)alkylene or (C3-C30)alkenylene with or without a fused ring to form a fused ring;

each one of the heterocycloalkyl and heteroaryl contains one or more heteroatom(s) selected from B, N, O, S, P(═O), Si and P.

The electroluminescent layer means the layer where electroluminescence occurs, and it may be a single layer or a multi-layer consisting of two or more layers laminated. When a mixture of host-dopant is used according to the constitution of the present invention, noticeable improvement in luminous efficiency due to the inventive electroluminescent host could be confirmed. This can be achieved by the doping concentration of 0.5 to 10% by weight. The host according to the present invention exhibits higher hole and electron conductivity, and excellent stability of material as compared to other conventional host materials, and provides improved device life as well as luminous efficiency.

The dopant compounds represented by Chemical Formula (2) or (3) can be exemplified by those described in Korean Patent Application No. 10-2009-0023442. More preferably they are selected from the following structures, but not restricted thereto.

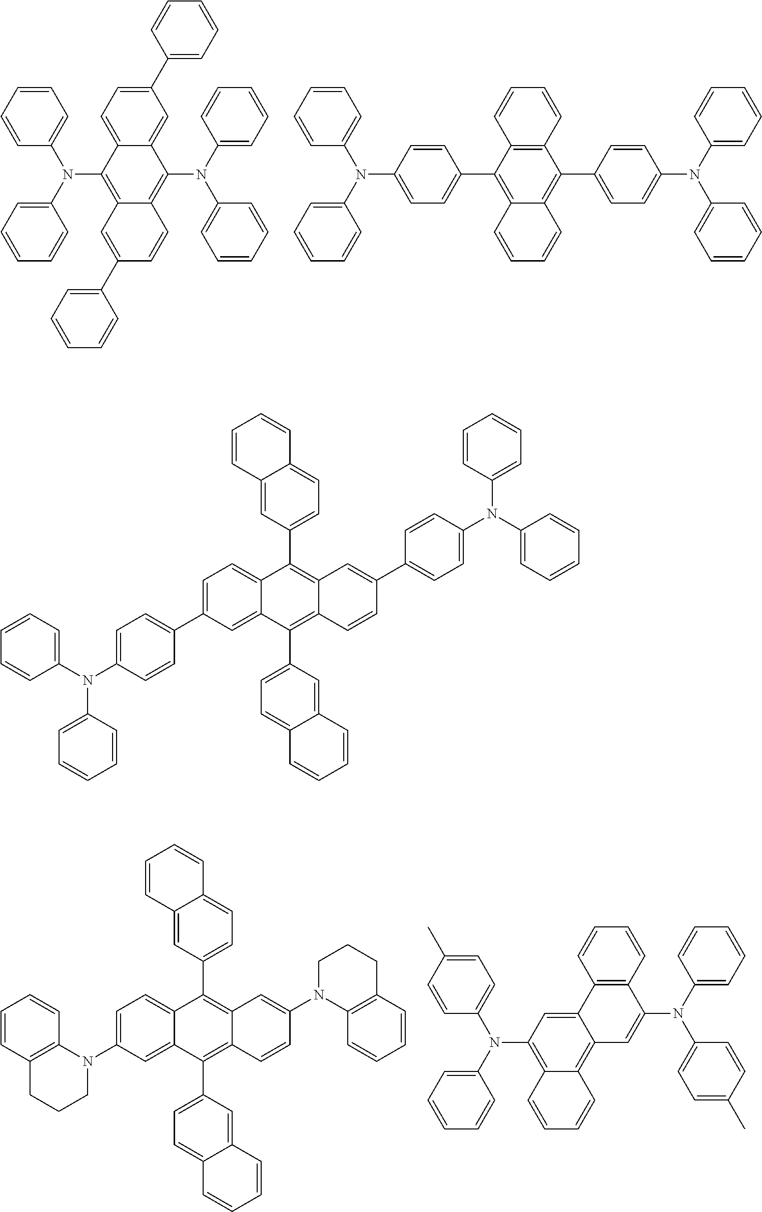

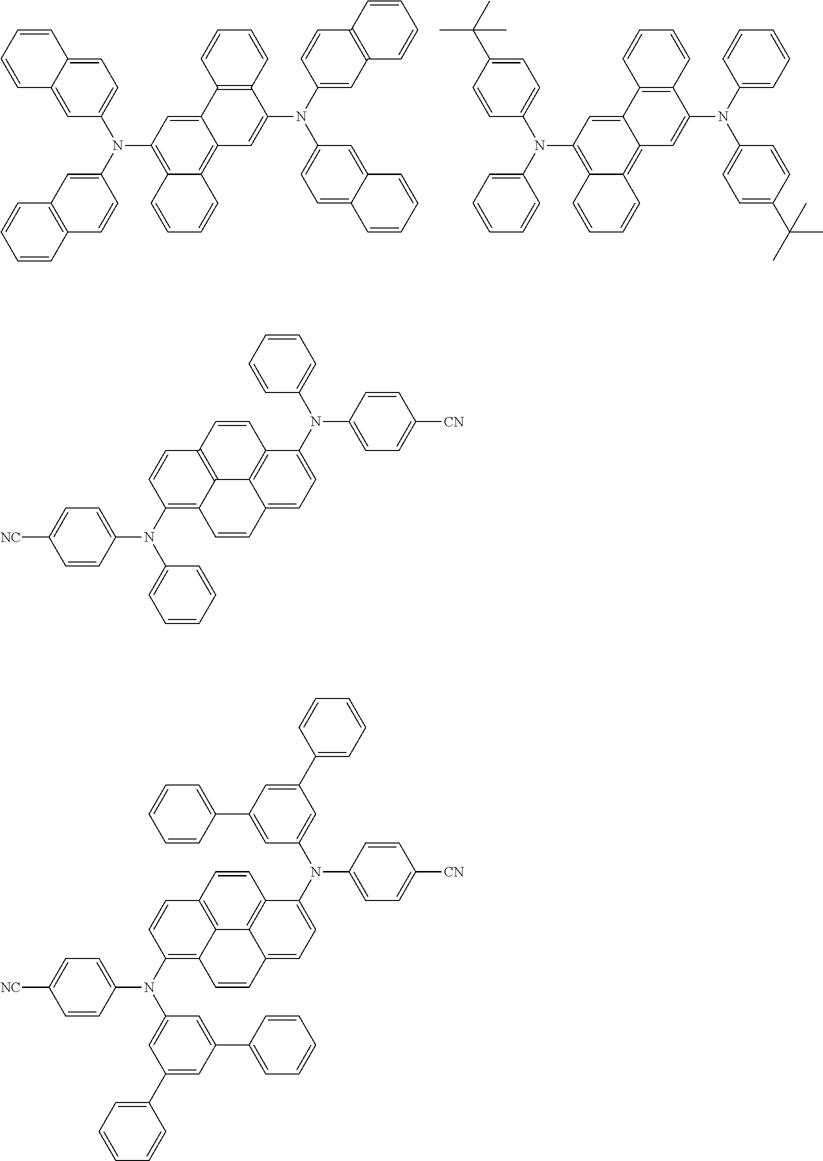

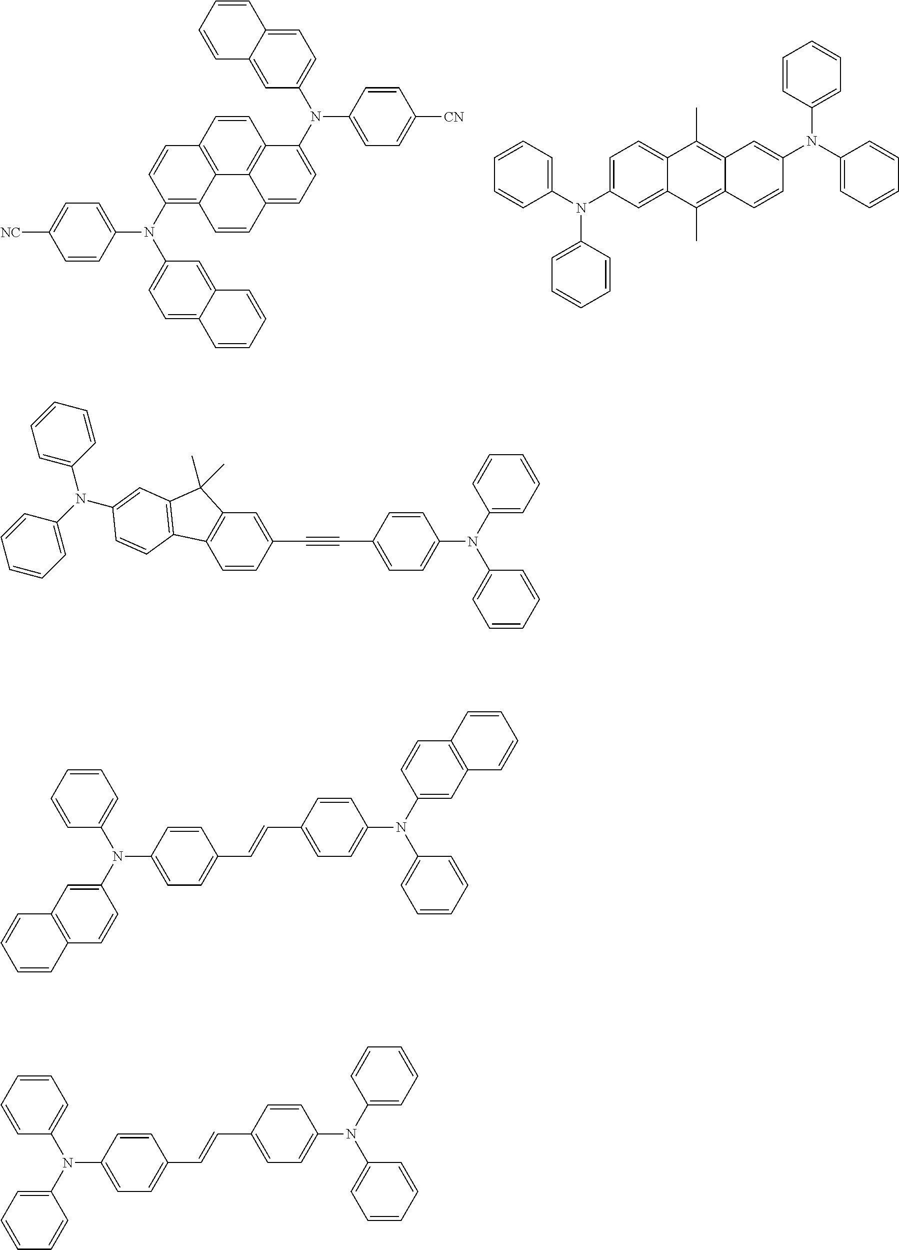

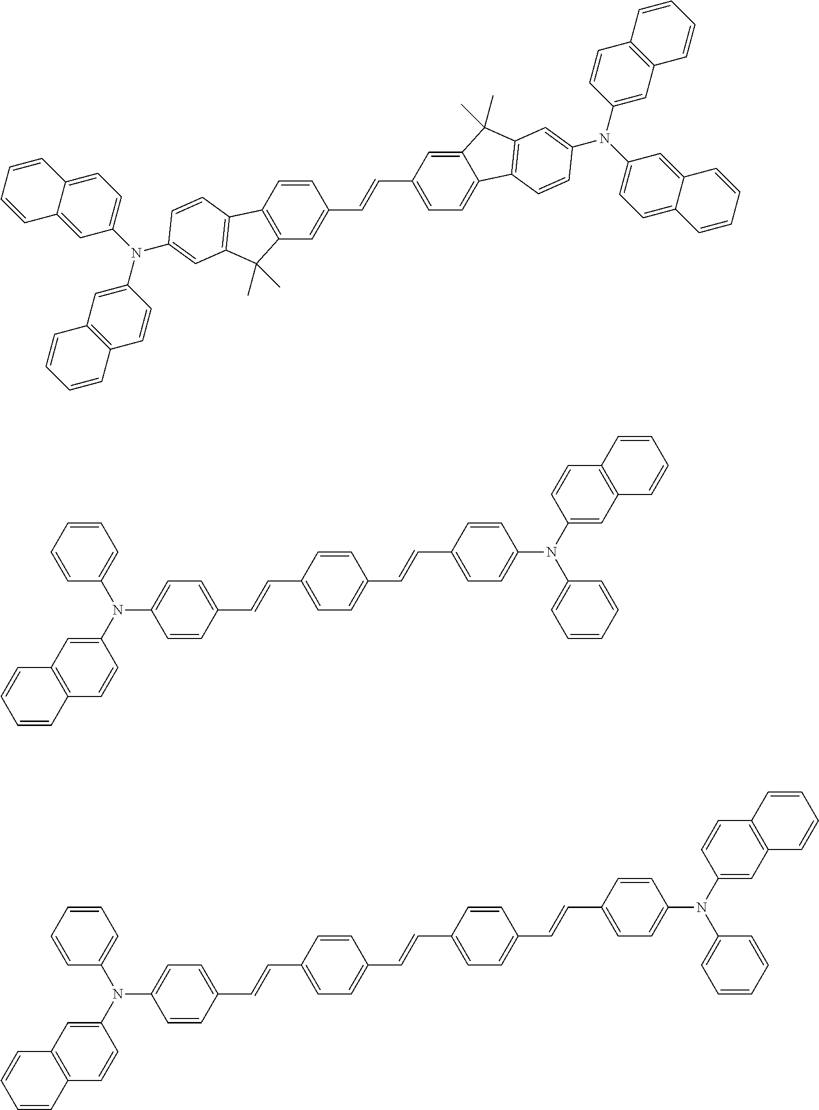

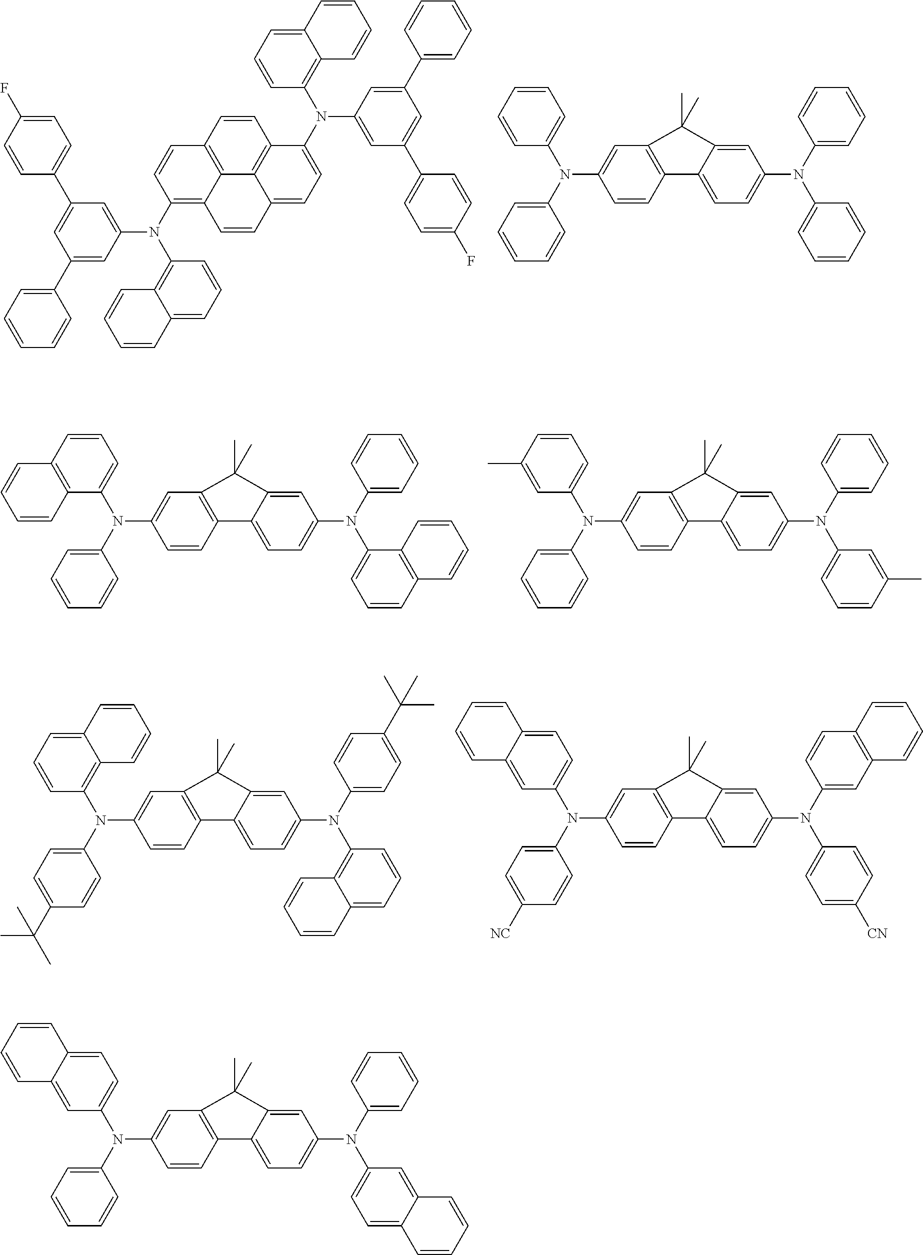

The organic electroluminescent device according to the present invention may further comprise one or more compound(s) selected from a group consisting of arylamine compounds and styrylarylamine compounds, in addition to the organic electroluminescent compound represented by Chemical Formula (1). The arylamine or styrylarylamine compounds are exemplified in Korean Patent Application Nos. 10-2008-0123276, 10-2008-0107606 and 10-2008-0118428, but not being restricted thereto.

In an organic electroluminescent device according to the present invention, the organic layer may further comprise one or more metal(s) selected from a group consisting of organometals of Group 1, Group 2, 4^{th} period and 5^{th} period transition metals, lanthanide metals and d-transition elements in the Periodic Table of Elements, or complex(es) thereof, as well as the organic electroluminescent compound represented by Chemical Formula (1). The organic layer may comprise an electroluminescent layer and a charge generating layer at the same time.

The organic electroluminescent device may also comprise one or more organic electroluminescent layer(s) emitting blue, green or red light, in addition to the organic electroluminescent compound (s) represented by Chemical Formula (1), to form an organic electroluminescent device emitting white light. The compounds emitting blue, green or red light are exemplified by Korean Patent Application Nos. 10-2008-0123276, 10-2008-0107606 and 10-2008-0118428, but not being restricted thereto.

In an organic electroluminescent device according to the present invention, it is preferable to arrange one or more layer(s) (here-in-below, referred to as the ‘surface layer’) selected from chalcogenide layers, metal halide layers and metal oxide layers, on the inner surface of at least one side of the pair of electrodes. Specifically, it is preferable to arrange a chalcogenide layer of silicon and aluminum metal (including oxides) on the anode surface of the electroluminescent medium layer, and a metal halide layer or a metal oxide layer on the cathode surface of the EL medium layer. As the result, stability in operation can be obtained.

Examples of chalcogenides preferably include SiOₓ (1≦X≦2), AlOₓ (1≦X≦1.5), SiON, SiAlON, or the like. Examples of metal halides preferably include LiF, MgF₂, CaF₂, fluorides of rare earth metal or the like. Examples of metal oxides preferably include Cs₂O, Li₂O, MgO, SrO, BaO, CaO, or the like.

In an organic electroluminescent device according to the present invention, it is also preferable to arrange, on at least one surface of the pair of electrodes thus manufactured, a mixed region of electron transport compound and a reductive dopant, or a mixed region of a hole transport compound with an oxidative dopant. Accordingly, the electron transport compound is reduced to an anion, so that injection and transportation of electrons from the mixed region to an EL medium are facilitated. In addition, since the hole transport compound is oxidized to form a cation, injection and transportation of holes from the mixed region to an EL medium are facilitated. Preferable oxidative dopants include various Lewis acids and acceptor compounds. Preferable reductive dopants include alkali metals, alkali metal compounds, alkaline earth metals, rare-earth metals, and mixtures thereof.

A white electroluminescent device having two or more electroluminescent layers can be manufactured by employing a reductive dopant layer as a charge generating layer.

### Advantageous Effects

The organic electroluminescent compounds according to the present invention exhibit high luminous efficiency and excellent life property of the material, so that OLED's having very good operation life can be manufactured therefrom.

### Best Mode

The present invention is further described by referring to representative compounds with regard to the organic electroluminescent compounds according to the invention, preparation thereof and luminescent properties of the devices manufactured therefrom, but those examples are provided for illustration of the embodiments only, not being intended to limit the scope of the invention by any means.

### PREPARATION EXAMPLE 1

### Preparation of Compound (1)

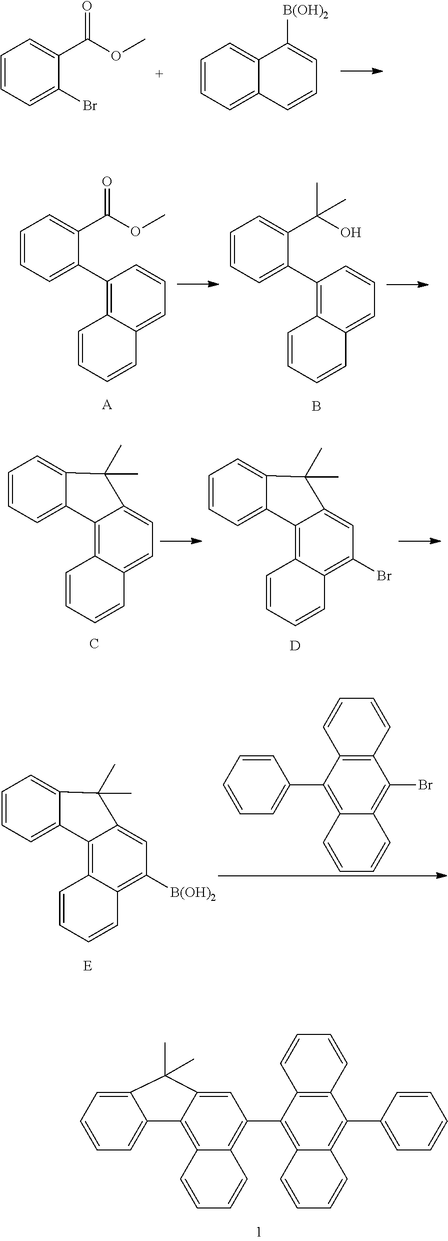

Preparation of Compound (A)

A two-necked flask was charged with methyl 2-bromobenzoate (40 g, 152.6 mmol), naphthalen-1-ylboronic acid (31.5 g, 183.2 mmol) and tetrakis(triphenylphophine)palladium [Pd(PPh₃)₄] (8.8 g, 7.62 mmol). While stirring the mixture, toluene (1 L) was added, and then 2 M potassium carbonate solution (228 mL, 458 mmol) and ethanol (228 mL). The mixture was heated under reflux at 100° C. for 5 hours. When the reaction was completed, the reaction mixture was cooled to room temperature, and extracted with distilled water and ethyl acetate. The organic layer was dried over MgSO₄, and evaporated by using a rotary evaporator to remove solvent. Purification via column chromatography (hexane and ethyl acetate as eluent) gave Compound (A) (35 g, 87%).

Preparation of Compound (B)

A one-necked flask containing Compound (A) (24 g, 91.49 mmol) was vacuated and filled with argon. After adding tetrahydrofuran (1 L), the mixture was stirred at −75° C. for 10 minutes. Methyl lithium (1.6 M MeLi in hexane) (257 mL, 0.41 mol) was added thereto, and the resultant mixture was stirred at −75° C. for 10 minutes, and then at ambient temperature for 3 hours. When the reaction was completed, the reaction mixture was extracted with distilled water and ethyl acetate. The organic layer was dried over MgSO₄, and evaporated by using a rotary evaporator to remove solvent. Purification via column chromatography (hexane and ethyl acetate as eluent) gave Compound (B) (20 g, 83%) .

Preparation of Compound (C)

To a one-necked flask containing Compound (B) (20 g, 76.23 mmol), added was AcOH (300 mL), and the mixture was stirred at 0° C. for 10 minutes. After adding H₃PO₄ (400 mL), the resultant mixture was stirred at ambient temperature for 1 hour. When the reaction was completed, the reaction mixture was neutralized by adding NaOH, and extracted with distilled water and ethyl acetate. The organic layer was dried over MgSO₄, and evaporated by using a rotary evaporator to remove solvent. Purification via column chromatography (hexane and ethyl acetate as eluent) gave Compound (C) (13.5 g, 720).

Preparation of Compound (D)

A one-necked flask containing Compound (C) (13.5 g, 55.25 mmol) was vacuated and filled with argon. After adding tetrahydrofuran (500 mL), the mixture was stirred at 0° C. for 10 minutes. NBS (19.6 g, 0.11 mol) was added thereto, and the resultant mixture was stirred at ambient temperature for one day. When the reaction was completed, the reaction mixture was extracted with distilled water and ethyl acetate. The organic layer was dried over MgSO₄, and evaporated by using a rotary evaporator to remove solvent. Purification via column chromatography (hexane and ethyl acetate as eluent) gave Compound (D) (13 g, 73%).

Preparation of Compound (E)

A one-necked flask containing Compound (D) (13 g, 42.21 mmol) was vacuated and filled with argon. After adding tetrahydrofuran (500 mL), the mixture was stirred at −78° C. for 10 minutes. To the mixture added was n-BuLi (2.5 M in hexane) (24. 1 mL, 60.32 mmol), and the resultant mixture was stirred at the same temperature for 1.5 hours. Trimethyl borate (6.85 mL, 60.32 mmol) was then added at −78° C. The reaction mixture was stirred at the same temperature for 30 minutes and then at room temperature for 4 hours. When the reaction was completed, the reaction mixture was extracted with distilled water and ethyl acetate. The organic layer was dried over MgSO₄, and evaporated by using a rotary evaporator to remove solvent. Purification via column chromatography (hexane and ethyl acetate as eluent) gave Compound (E) (8 g, 69%).

Preparation of Compound (1)

A mixture of Compound (D) (5.0 g, 13.4 mmol), 9-phenyl-anthracene-10-boronic acid (4.8 g, 16.1 mmol), Pd(PPh₃)₄ (0.8 g, 0.7 mmol), aqueous 2 M K₂CO₃ solution (20 mL), toluene (100 mL) and ethanol (50 mL) was stirred under reflux for 12 hours. When the reaction was completed, the reaction mixture was extracted with distilled water and ethyl acetate. The extract was dried over magnesium sulfate and distilled under reduced pressure. Purification via column chromatography gave Compound (1) (4.3 g, 7.9 mmol, 58.8%).

According to the same procedure as described in Preparation Example 1, prepared were organic electroluminescent compounds (Compounds 1 to 150), of which ¹H NMR and MS/FAB data are listed in Table 1.

TABLE 1

| | | MS/FAB | |
|---|---|---|---|
| Com- | | | calcu- |
| pound | ¹H NMR (CDCl₃, 200 MHz) | found | lated |
| 1 | δ = 1.78 (6H, s), 7.24 (1H, m), 7.39~7.44 (6H, | 496.64 | 496.22 |
| | m), 7.51~7.54 (6H, m), 7.61 (1H, m), 7.91 (4H, | | |
| | m), 7.99 (1H, s), 8.09 (1H, m), 8.52~8.56 (2H, | | |
| | m) | | |
| 2 | δ = 1.78 (6H, s), 7.24 (1H, m), 7.39~7.44 (5H, | 546.70 | 546.23 |
| | m), 7.51~7.61 (6H, m), 7.73 (1H, m), | | |
| | 7.91~7.92 (5H, m), 7.99 (1H, s), 8 (2H, m), | | |
| | 8.09 (1H, m), 8.52~8.56 (2H, m) | | |
| 4 | δ = 1.78 (6H, s), 7.24 (1H, m), 7.39~7.44 (5H, | 596.76 | 596.25 |
| | m), 7.51~7.54 (2H, m), 7.61 (1H, m), | | |
| | 7.82~7.93 (9H, m), 7.99 (1H, s), 8.09~8.12 | | |
| | (3H, m), 8.52~8.56 (2H, m), 8.93 (2H, m) | | |
| 6 | δ = 1.72 (6H, s), 1.78 (6H, s), 7.24~7.28 (2H, | 612.80 | 612.28 |
| | m), 7.38~7.44 (6H, m), 7.51~7.55 (3H, m), | | |
| | 7.61~7.63 (2H, m), 7.77 (1H, m), 7.87~7.93 | | |
| | (6H, m), 7.99 (1H, s), 8.09 (1H, m), 8.52~8.56 | | |
| | (2H, m) | | |
| 10 | δ = 1.78 (6H, s), 7.24 (1H, m), 7.39~7.44 (7H, | 648.83 | 648.28 |
| | m), 7.51~7.54 (10H, m), 7.61~7.66 (4H, m), | | |
| | 7.91 (4H, m), 7.99 (1H, s), 8.09 (1H, m), | | |
| | 8.52~8.56 (2H, m) | | |
| 16 | δ = 1.78 (6H, s), 7.24 (1H, m), 7.39~7.44 (6H, | 622.79 | 622.27 |
| | m), 7.51~7.61 (7H, m), 7.79 (2H, m), 7.91 (4H, | | |
| | m), 7.99 (1H, s), 8 (2H, m), 8.09 (1H, m), 8.4 | | |
| | (2H, m), 8.52~8.56 (2H, m) | | |
| 19 | δ = 1.78 (6H, s), 7.24 (1H, m), 7.39~7.44 (5H, | 521.65 | 521.21 |
| | m), 7.51~7.54 (2H, m), 7.61 (1H, m), | | |
| | 7.82~7.84 (4H, m), 7.91 (4H, m), 7.99 (1H, s), | | |
| | 8.09 (1H, m), 8.52~8.56 (2H, m) | | |
| 24 | δ = 1.78 (6H, s), 7.24 (1H, m), 7.37~7.46 | 755.03 | 754.31 |
| | (24H, m), 7.61 (1H, m), 7.89~7.91 (6H, m), | | |
| | 7.99 (1H, s), 8.09 (1H, m), 8.52~8.56 (2H, m) | | |
| 26 | δ = 1.78 (6H, s), 7.24 (1H, m), 7.39~7.54 (9H, | 602.78 | 602.21 |
| | m), 7.61 (1H, m), 7.86~7.91 (5H, m), 7.98 (1H, | | |
| | m), 7.99 (1H, s), 8 (2H, m), 8.09 (1H, m), 8.45 | | |
| | (1H, m), 8.52~8.56 (2H, m) | | |
| 28 | δ = 1.78 (6H, s), 7.24~7.25 (2H, m), 7.33 (1H, | 661.83 | 661.28 |
| | m), 7.39~7.5 (13H, m), 7.69 (1H, m), 7.77 (1H, | | |
| | m), 7.87~7.94 (6H, m), 7.99 (1H, s), 8.09 (1H, | | |
| | m), 8.52~8.56 (3H, m) | | |
| 35 | δ = 1.78 (6H, s), 7.1 (1H, m), 7.24 (1H, m), | 547.69 | 547.23 |
| | 7.39~7.54 (9H, m), 7.61~7.63 (2H, m), | | |
| | 7.91~7.92 (5H, m), 7.99 (1H, s), 8.09 (1H, m), | | |
| | 8.42 (1H, m), 8.52~8.56 (2H, m) | | |
| 39 | δ = 1.78 (6H, s), 7.24~7.33 (4H, m), 7.4~7.54 | 585.73 | 585.25 |
| | (8H, m), 7.61~7.63 (2H, m), 7.9~7.94 (5H, m), | | |
| | 7.99 (1H, s), 8.09~8.12 (2H, m), 8.52~8.56 | | |
| | (3H, m) | | |
| 46 | δ = 1.78 (6H, s), 7.11 (4H, m), 7.24~7.44 | 736.94 | 736.31 |
| | (14H, m), 7.51~7.55 (3H, m), 7.61~7.63 (2H, | | |
| | m), 7.77 (1H, m), 7.87~7.93 (6H, m), 7.99 (1H, | | |
| | s), 8.09 (1H, m), 8.52~8.56 (2H, m) | | |
| 47 | δ = 1.78 (12H, s), 7.14 (1H, m), 7.24 (1H, m), | 662.86 | 662.30 |
| | 7.39~7.44 (5H, m), 7.51~7.54 (4H, m), 7.61 | | |
| | (1H, m), 7.69 (1H, m), 7.83 (1H, m), 7.91 (4H, | | |
| | m), 7.98 (1H, m), 7.99 (1H, s), 8.09 (2H, m), | | |
| | 8.15 (1H, m), 8.52~8.56 (3H, m) | | |
| 48 | δ = 1.78 (12H, s), 7.24 (2H, m), 7.39~7.44 | 662.86 | 662.30 |
| | (6H, m), 7.51~7.54 (4H, m), 7.61 (2H, m), 7.91 | | |
| | (4H, m), 7.99 (2H, s), 8.09 (2H, m), 8.52~8.56 | | |
| | (4H, m) | | |
| 54 | δ = 1.78 (6H, s), 7.24 (1H, m), 7.25~7.39 | 661.83 | 661.28 |
| | (11H, m), 7.61~7.68 (4H, m), 7.79 (2H, m), | | |
| | 7.91~7.94 (5H, m), 7.99 (1H, s), 8.09~8.12 | | |
| | (2H, m), 8.52~8.56 (3H, m) | | |
| 59 | δ = 0.14 (6H, s), 1.78 (6H, s), 5.2 (1H, m), 6.68 | 528.76 | 528.23 |
| | (1H, s), 7.04 (1H, s), 7.24 (1H, m), 7.39~7.44 | | |
| | (5H, m), 7.51~7.54 (2H, m), 7.61 (1H, m), 7.91 | | |
| | (4H, m), 7.99 (1H, s), 8.09 (1H, m), 8.52~8.56 | | |
| | (2H, m) | | |
| 62 | δ = 1.72 (12H, s), 1.78 (6H, s), 7.24 (2H, m), | 728.96 | 728.34 |
| | 7.39~7.44 (6H, m), 7.51~7.54 (2H, m), | | |
| | 7.61~7.63 (3H, m), 7.69 (1H, s), 7.77 (2H, s), | | |
| | 7.77 (0H, m), 7.91~7.93 (5H, m), 7.99 (1H, s), | | |
| | 8.09 (2H, m), 8.52~8.56 (2H, m) | | |
| 64 | δ = 1.78 (6H, s), 1.91 (6H, s), 7.24 (1H, m), | 712.92 | 712.31 |
| | 7.39~7.44 (5H, m), 7.51~7.54 (6H, m), 7.61 | | |
| | (1H, m), 7.82 (1H, m), 7.91 (4H, m), 7.98 (1H, | | |
| | m), 7.99 (2H, s), 8.05~8.09 (2H, m), 8.18 (1H, | | |
| | m), 8.52~8.56 (4H, m) | | |
| 65 | δ = 1.78 (6H, s), 1.85 (6H, s), 7.24 (1H, m), | 662.86 | 662.30 |
| | 7.39~7.44 (5H, m), 7.51~7.54 (4H, m), 7.61 | | |
| | (1H, m), 7.69 (1H, m), 7.82~7.83 (2H, m), 7.91 | | |
| | (4H, m), 7.98 (1H, m), 7.99 (1H, s), 8.05~8.09 | | |
| | (2H, m), 8.15~8.18 (2H, m), 8.52~8.56 (2H, | | |
| | m) | | |
| 66 | δ = 1.78 (6H, s), 1.84 (6H, s), 7.24 (1H, m), | 712.92 | 712.31 |
| | 7.39~7.44 (5H, m), 7.51~7.61 (8H, m), | | |
| | 7.91~7.97 (5H, m), 7.99 (2H, s), 8.01 (1H, m), | | |
| | 8.09 (1H, m), 8.28 (1H, m), 8.52~8.56 (4H, m) | | |
| 68 | δ = 1.78 (12H, s), 7.24 (1H, m), 7.39~7.44 | 662.86 | 662.30 |
| | (5H, m), 7.51~7.61 (6H, m), 7.69 (1H, m), 7.83 | | |
| | (1H, m), 7.91~7.97 (5H, m), 7.99 (1H, s), 8.01 | | |
| | (1H, m), 8.09 (1H, m), 8.15 (1H, m), 8.28 (1H, | | |
| | m), 8.52~8.56 (2H, m) | | |
| 69 | δ = 1.78 (6H, s), 1.84 (6H, s), 7.14 (1H, m), | 712.92 | 712.31 |
| | 7.24 (1H, m), 7.39~7.44 (5H, m), 7.51~7.54 | | |
| | (6H, m), 7.61 (1H, m), 7.91 (4H, m), 7.98 (1H, | | |
| | m), 7.99 (2H, s), 8.09 (2H, m), 8.52~8.56 (5H, | | |
| | m) | | |
| 70 | δ = 1.78 (12H, s), 7.14 (1H, m), 7.24 (2H, m), | 662.86 | 662.30 |
| | 7.39~7.44 (6H, m), 7.51~7.54 (2H, m), 7.61 | | |
| | (2H, m), 7.9~7.98 (7H, m), 7.99 (1H, s), | | |
| | 8.09~8.11 (3H, m), 8.52~8.56 (2H, m) | | |
| 71 | δ = 1.78 (6H, s), 1.96 (2H, m), 2.76 (2H, m), | 551.72 | 551.26 |
| | 3.06 (2H, m), 6.55 (1H, m), 6.72 (1H, m), | | |
| | 7.05~7.07 (2H, m), 7.24 (1H, m), 7.38 (4H, m), | | |
| | 7.44 (1H, m), 7.51~7.54 (2H, m), 7.61 (1H, m), | | |
| | 7.88~7.9 (4H, m), 7.99 (1H, s), 8.09 (1H, m), | | |
| | 8.52~8.56 (2H, m) | | |
| 73 | δ = 1.78 (6H, s), 3.81 (2H, s), 6.51 (2H, m), | 599.76 | 599.26 |
| | 6.69 (2H, m), 6.98~7.01 (4H, m), 7.24 (1H, m), | | |
| | 7.38 (4H, m), 7.44 (1H, m), 7.51~7.54 (2H, m), | | |
| | 7.61 (1H, m), 7.88~7.9 (4H, m), 7.99 (1H, s), | | |
| | 8.09 (1H, m), 8.52~8.56 (2H, m) | | |
| 85 | δ = 1.72 (6H, s), 1.78 (6H, s), 6.55 (1H, m), | 703.91 | 703.32 |
| | 6.61~6.63 (3H, m), 6.73 (1H, m), 6.81 (1H, m), | | |
| | 7.02~7.05 (2H, m), 7.2~7.24 (3H, m), | | |
| | 7.36~7.44 (6H, m), 7.51~7.54 (2H, m), 7.61 | | |
| | (2H, m), 7.91 (4H, m), 7.99 (1H, s), 8.09 (1H, | | |
| | m), 8.52~8.56 (2H, m) | | |
| 90 | δ = 1.78 (6H, s), 7.24~7.25 (5H, m), | 622.79 | 622.27 |
| | 7.39~7.44 (5H, m), 7.51~7.55 (4H, m), 7.61 | | |
| | (2H, m), 7.91 (4H, m), 7.99 (1H, s), 8.04~8.09 | | |
| | (3H, m), 8.42 (1H, m), 8.52~8.56 (3H, m) | | |
| 93 | δ = 1.78 (6H, s), 7.24 (1H, m), 7.39~7.55 | 622.79 | 622.27 |
| | (11H, m), 7.61 (2H, m), 7.85 (2H, m), 7.91 | | |
| | (4H, m), 7.99 (1H, s), 8.04~8.09 (3H, m), 8.42 | | |
| | (1H, m), 8.52~8.56 (3H, m) | | |
| 95 | δ = 1.78 (6H, s), 7.24 (1H, m), 7.39~7.44 | 672.85 | 672.28 |
| | (10H, m), 7.51~7.54 (6H, m), 7.61 (1H, m), | | |
| | 7.91 (8H, m), 7.99 (1H, s), 8.09 (1H, m), | | |
| | 8.52~8.56 (2H, m) | | |
| 97 | δ = 1.78 (6H, s), 7.24 (1H, m), 7.39~7.55 | 799.01 | 798.33 |
| | (15H, m), 7.61 (2H, m), 7.85 (2H, m), 7.91 | | |
| | (8H, m), 7.99 (1H, s), 8.04~8.09 (3H, m), 8.42 | | |
| | (1H, m), 8.52~8.56 (3H, m) | | |
| 100 | δ = 1.78 (6H, s), 7.24~7.25 (5H, m), 7.39~7.44 | 748.95 | 748.31 |
| | (10H, m), 7.51~7.54 (6H, m), 7.61 (1H, m), | | |
| | 7.91 (8H, m), 7.99 (1H, s), 8.09 (1H, m), | | |
| | 8.52~8.56 (2H, m) | | |
| 104 | δ = 1.78 (6H, s), 7.24 (1H, m), 7.39~7.44 (6H, | 622.79 | 622.27 |
| | m), 7.51~7.61 (9H, m), 7.73 (2H, m), | | |
| | 7.91~7.92 (6H, m), 7.99 (1H, s), 8.09 (1H, m), | | |
| | 8.52~8.56 (2H, m) | | |
| 105 | δ = 1.78 (6H, s), 7.24 (1H, m), 7.39~7.44 (6H, | 622.79 | 622.27 |
| | m), 7.51~7.54 (4H, m), 7.61 (3H, m), 7.79 (2H, | | |
| | m), 7.91 (4H, m), 7.99 (1H, s), 8.09 (1H, m), | | |
| | 8.42 (2H, m), 8.51~8.56 (4H, m) | | |
| 109 | δ =1.78 (6H, s), 6.63 (4H, m), 6.69 (2H, m), | 663.85 | 663.29 |
| | 6.81 (2H, m), 7.2~7.24 (5H, m), 7.39~7.44 | | |
| | (5H, m), 7.51~7.54 (4H, m), 7.61 (1H, m), 7.91 | | |
| | (4H, m), 7.99 (1H, s), 8.09 (1H, m), 8.52~8.56 | | |
| | (2H, m) | | |
| 110 | δ = 1.78 (6H, s), 7.11 (6H, m), 7.24~7.44 | 738.95 | 738.33 |
| | (19H, m), 7.51~7.54 (2H, m), 7.61 (1H, m), | | |
| | 7.91 (4H, m), 7.99 (1H, s), 8.09 (1H, m), | | |
| | 8.52~8.56 (2H, m) | | |
| 111 | δ = 1.78 (6H, s), 7.24 (1H, m), 7.39~7.44 (5H, | 600.75 | 600.25 |
| | m), 7.51~7.55 (4H, m), 7.61~7.64 (2H, m), | | |
| | 7.74~7.84 (6H, m), 7.91 (4H, m), 7.99 (1H, s), | | |
| | 8.09 (1H, m), 8.52~8.56 (2H, m) | | |
| 114 | δ = 1.78 (6H, s), 6.63 (4H, m), 6.81 (2H, m), | 587.75 | 587.26 |
| | 7.2~7.24 (5H, m), 7.38 (4H, m), 7.44 (1H, m), | | |
| | 7.51~7.54 (2H, m), 7.61 (1H, m), 7.88~7.9 | | |
| | (4H, m), 7.99 (1H, s), 8.09 (1H, m), 8.52~8.56 | | |
| | (2H, m) | | |
| 119 | δ = 1.78 (6H, s), 7.24 (1H, m), 7.39 (8H, m), | 748.95 | 748.31 |
| | 7.41~7.51 (12H, m), 7.7 (1H, m), 7.91 (8H, m), | | |
| | 7.99 (1H, s), 8.09 (1H, m), 8.52~8.56 (2H, m) | | |
| 120 | δ = 1.78 (6H, s), 7.24 (1H, m), 7.35~7.44 (6H, | 598.73 | 598.24 |
| | m), 7.51~7.61 (4H, m), 7.81 (1H, m), 7.91 (4H, | | |
| | m), 7.99 (1H, s), 8.06~8.1 (3H, m), 8.38 (1H, | | |
| | m), 8.52~8.56 (2H, m), 8.83 (1H, m) | | |
| 121 | δ = 1.78 (6H, s), 3.05 (2H, m), 4.14 (2H, m), | 537.69 | 537.25 |
| | 6.55 (1H, m), 6.72 (1H, m), 7.05~7.07 (2H, m), | | |
| | 7.24 (1H, m), 7.38 (4H, m), 7.44 (1H, m), | | |
| | 7.51~7.54 (2H, m), 7.61 (1H, m), 7.88~7.9 | | |
| | (4H, m), 7.99 (1H, s), 8.09 (1H, m), 8.52~8.56 | | |
| | (2H, m) | | |
| 125 | δ = 1.78 (6H, s), 7.15 (1H, m), 7.32 (1H, m), | 569.69 | 569.23 |
| | 7.39 (4H, m), 7.51~7.59 (5H, m), 7.73 (1H, m), | | |
| | 7.91~7.92 (5H, m), 7.99 (1H, s), 8 (2H, m), | | |
| | 8.07 (1H, m), 8.52~8.56 (2H, m) | | |
| 129 | δ = 1.78 (6H, s), 7.15 (1H, m), 7.32 (1H, m), | 690.84 | 690.27 |
| | 7.39~7.41 (9H, m), 7.51~7.54 (6H, m), 7.91 | | |
| | (8H, m), 7.99 (1H, s), 8.07 (1H, m), 8.52~8.56 | | |
| | (2H, m) | | |
| 133 | δ = 1.72 (6H, s), 7.15 (1H, m), 7.32 (1H, m) | 515.62 | 515.20 |
| | 7.39~7.41 (5H, m), 7.51~7.54 (5H, m), 7.76 | | |
| | (1H, s), 7.91 (4H, m), 8.07 (1H, m), 8.39 (1H, | | |
| | m), 8.82 (1H, m) | | |
| 134 | δ = 1.72 (6H, s), 2.34 (3H, s), 7.24 (1H, m), | 511.65 | 511.23 |
| | 7.39~7.41 (6H, m), 7.51~7.54 (5H, m), 7.76 | | |
| | (1H, s), 7.91~7.97 (5H, m), 8.39 (1H, m), 8.82 | | |
| | (1H, m) | | |
| 135 | δ = 1.72 (6H, s), 7.24 (1H, m), 7.39~7.44 (6H, | 497.63 | 497.21 |
| | m), 7.51~7.54 (5H, m), 7.61 (1H, m), 7.76 (1H, | | |
| | s), 7.91 (4H, m), 8.09 (1H, m), 8.39 (1H, m), | | |
| | 8.82 (1H, m) | | |
| 136 | δ = 1.78 (6H, s), 7.39~7.41 (5H, m), | 497.63 | 497.21 |
| | 7.51~7.54 (6H, m), 7.66 (1H, m), 7.91 (4H, m), | | |
| | 7.99 (1H, s), 8.47~8.56 (3H, m), 8.95 (1H, m) | | |
| 137 | δ = 1.78 (6H, s), 7.24 (1H, m), 7.39~7.54 | 660.65 | 660.30 |
| | (13H, m), 7.61 (1H, m), 7.75~7.81 (8H, m), | | |
| | 7.91 (4H, m), 7.99 (1H, s), 8.09 (1H, m), | | |
| | 8.52~8.56 (2H, m) | | |
| 138 | δ = 1.72 (6H, s), 7.33~7.41 (9H, m), | 497.63 | 497.21 |
| | 7.48~7.52 (5H, m), 7.71~7.73 (2H, m), | | |
| | 7.91~7.93 (5H, m) | | |
| 139 | δ = 1.78 (6H, s), 6.63 (6H, m), 6.81 (2H, m), | 689.88 | 689.31 |
| | 6.95 (2H, m), 7.2~7.24 (5H, m), 7.39~7.44 | | |
| | (5H, m), 7.51~7.54 (2H, m), 7.61 (1H, m), 7.77 | | |
| | (2H, m), 7.91 (4H, m), 7.99 (1H, s), 8.09 (1H, | | |
| | m), 8.52~8.56 (2H, m) | | |
| 141 | δ = 1.78 (6H, s), 6.95 (2H, m), 7.24 (1H, m), | 686.69 | 686.31 |
| | 7.39~7.54 (15H, m), 7.61 (1H, m), 7.75 (6H, | | |
| | m), 7.91 (4H, m), 7.99 (1H, s), 8.09 (1H, m), | | |
| | 8.52~8.56 (2H, m) | | |
| 142 | δ = 1.78 (6H, s), 6.95 (2H, m), 7.24 (1H, m), | 684.67 | 684.30 |
| | 7.39~7.54 (13H, m), 7.61 (1H, m), 7.75~7.81 | | |
| | (6H, m), 7.91 (4H, m), 7.99 (1H, s), 8.09 (1H, | | |
| | m), 8.52~8.56 (2H, m) | | |
| 143 | δ = 1.78 (6H, s), 6.95 (2H, m), 7.24 (1H, m), | 687.87 | 687.29 |
| | 7.25~7.39 (13H, m), 7.61~7.63 (4H, m), | | |
| | 7.91~7.94 (5H, m), 7.99 (1H, s), 8.09~8.12 | | |
| | (2H, m), 8.52~8.56 (3H, m) | | |
| 144 | δ = 1.78 (6H, s), 6.59~6.63 (6H, m), 6.81 (2H, | 687.87 | 687.29 |
| | m), 7.2~7.24 (5H, m), 7.34~7.44 (7H, m), | | |
| | 7.51~7.54 (2H, m), 7.61 (1H, m), 7.91 (4H, m), | | |
| | 7.99 (1H, s), 8.09 (1H, m), 8.52~8.56 (2H, m) | | |
| 145 | δ = 1.72 (6H, s), 1.78 (12H, s), 7.24~7.28 (2H, | 779.02 | 778.36 |
| | m), 7.38~7.44 (6H, m), 7.51~7.55 (5H, m), | | |
| | 7.61 (1H, m), 7.69 (1H, s), 7.77 (1H, s), | | |
| | 7.87~7.91 (5H, m), 7.99 (2H, s), 8.09 (1H, m), | | |
| | 8.52~8.56 (4H, m) | | |
| 146 | δ = 1.78 (6H, s), 7.24 (1H, m), 7.39~7.44 (5H, | 501.67 | 501.25 |
| | m), 7.51~7.54 (2H, m), 7.61 (1H, m), 7.91 (4H, | | |
| | m), 7.99 (1H, s), 8.09 (1H, m), 8.52~8.56 (2H, | | |
| | m) | | |
| 147 | δ = 1.78 (6H, s), 7.24 (1H, m), 7.39 (4H, m), | 711.89 | 711.29 |
| | 7.44 (1H, m), 7.45 (1H, m), 7.5~7.58 (11H, m), | | |
| | 7.77 (1H, m), 7.91 (4H, m), 7.99 (1H, s), 8 | | |
| | (1H, m), 8.09 (1H, m), 8.16~8.18 (2H, m), | | |
| | 8.52~8.56 (3H, m) | | |
| 150 | δ = 1.78 (6H, s), 7.24 (1H, m), 7.39 (4H, m), | 711.89 | 711.29 |
| | 7.4 (1H, m), 7.44 (1H, m), 7.45~7.54 (11H, m), | | |
| | 7.77 (1H, m), 7.91 (4H, m), 7.99 (1H, s), 8 | | |
| | (1H, m), 8.09 (1H, m), 8.16~8.18 (3H, m), | | |
| | 8.52~8.56 (2H, m) | | |

### EXAMPLE 1

### Manufacture of OLED's by using Organic Electroluminescent Compounds According to the Present Invention

OLED devices were manufactured by using the electroluminescent material according to the invention.

First, a transparent electrode ITO thin film (15Ω/□) prepared from glass for OLED (produced by Samsung-Corning) was subjected to ultrasonic washing with trichloroethylene, acetone, ethanol and distilled water, sequentially, and stored in isopropanol before use.

Then, an ITO substrate was equipped in a substrate folder of a vacuum vapor-deposit device, and 4,4′,4″-tris(N,N-(2-naphthyl)-phenylamino)triphenylamine (2-TNATA) (of which the chemical structure is shown below) was placed in a cell of the vacuum vapor-deposit device, which was then ventilated to reach 10⁻⁶ torr of vacuum in the chamber. Electric current was applied to the cell to evaporate 2-TNATA, thereby providing vapor-deposit of a hole injection layer having 60 nm of thickness on the ITO substrate.

Then, to another cell of the vacuum vapor-deposit device, charged was N,N′-bis(α-naphthyl)-N,N′-diphenyl-4,4′-diamine (NPB), and electric current was applied to the cell to evaporate NPB, thereby providing vapor-deposit of a hole transport layer of 20 nm of thickness on the hole injection layer.

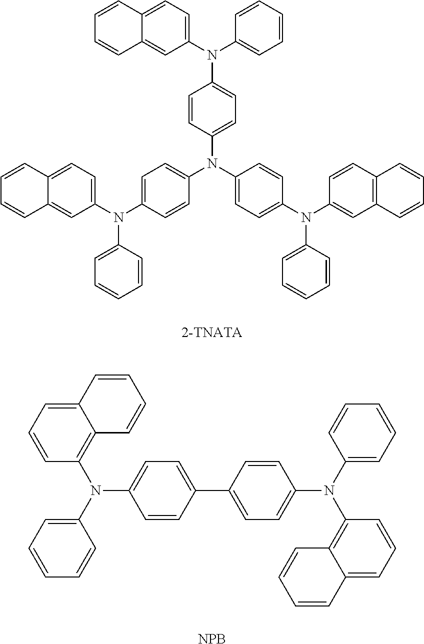

After forming the hole injecting layer and the hole transport layer, an electroluminescent layer was vapor-deposited thereon as follows. To one cell of a vacuum vapor-deposit device, charged was Compound (1) according to the present invention, and Compound (D) (of which the structure is shown below) was charged to another cell. Two cells were simultaneously heated with vapor-deposition rate of Compound (A) at a concentration of 2 to 5% by weight. Thus, an electroluminescent layer having the thickness of 30 nm was vapor-deposited on the hole transport layer.

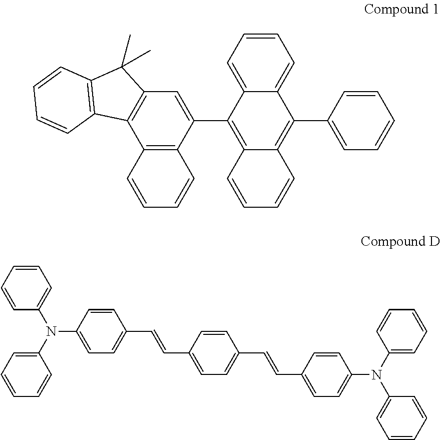

Then, tris(8-hydroxyquinoline)aluminum (III) (Alq) (of which the structure is shown below) was vapor-deposited as an electron transport layer with a thickness of 20 nm, and lithium quinolate (Liq) (of which the structure shown below) was vapor-deposited as an electron injecting layer with a thickness of 1 to 2 nm. Thereafter, an Al cathode was vapor-deposited with a thickness of 150 nm by using another vacuum vapor-deposit device to manufacture an OLED.

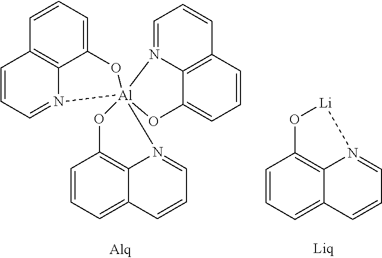

Each material employed for manufacturing an OLED was used as the electroluminescent material after purifying via vacuum sublimation at 10⁻⁶ torr.

### COMPARATIVE EXAMPLE 1

### Manufacture of an OLED by using Conventional Electroluminescent Compound

After forming a hole injecting layer and a hole transport layer according to the same procedure as described in Example 1, dinaphthylanthracene (DNA) was charged to another cell of said vacuum vapor-deposit device as electroluminescent host material, while Compound (D) was charged to still another cell as blue electroluminescent material. At a vapor-deposition rate of 100:1, an electroluminescent layer having the thickness of 30 nm was vapor-deposited on the hole transport layer.

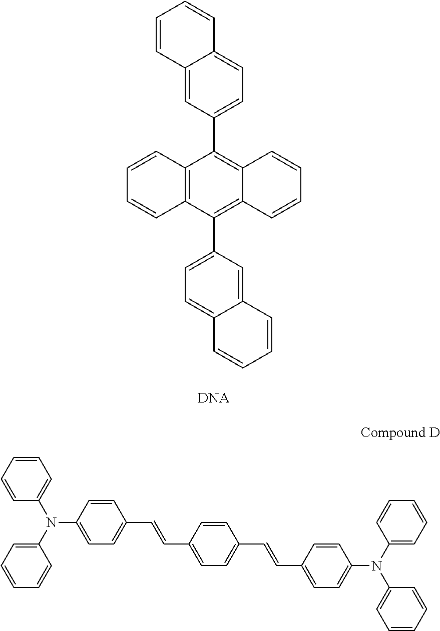

Then, an electron transport layer and an electron injecting layer were vapor-deposited according to the same procedures as in Example 1, and Al cathode was vapor-deposited by using another vacuum vapor-deposit device with a thickness of 150 nm, to manufacture an OLED.

The luminous efficiencies of the OLED's comprising the organic electroluminescent compounds according to the present invention (Example 1) and conventional electroluminescent compound (Comparative Example 1) were measured at 1,000 cd/m², respectively, and the results are shown in Table 2.

TABLE 2

| | | | | Doping | Luminous | |
|---|---|---|---|---|---|---|
| | | | | concen- | efficiency | |
| | | | | tration | (cd/A) | |
| No. | | Host | Dopant | (wt %) | @1000 cd/m² | Color |
| Ex. 1 | 1 | 1 | Comp. D | 3.0 | 12.5 | Jade green |
| | 2 | 2 | Comp. D | 3.0 | 12.7 | Jade green |
| | 3 | 40 | Comp. D | 3.0 | 12.6 | Jade green |
| | 4 | 48 | Comp. D | 3.0 | 12.8 | Jade green |
| | 5 | 128 | Comp. D | 3.0 | 12.7 | Jade green |

| | | | | | |
|---|---|---|---|---|---|
| Comp. Ex. 1 | DNA | Comp. D | 3.0 | 12.0 | Jade green |

As can be seen from Table 2, the blue electroluminescent devices manufactured by employing the electroluminescent material according to the present invention showed comparable or higher luminous efficiency, as compared to that of Comparative Example 1.

1. An organic electroluminescent compound represented by Chemical Formula (1): 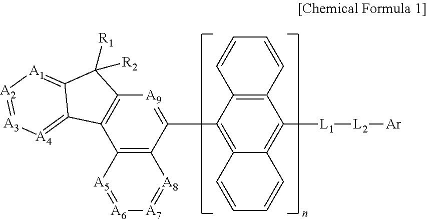
wherein
A₁ through A₉ independently represent CR₃₁ or N;
L₁ and L₂ independently represent a chemical bond,
(C6-C30)arylene with or without substituent(s),
(C3-C30)heteroarylene with or without substituent(s), 5- to 7-membered heterocycloalkylene with or without substituent(s), substituted or unsubstituted 5- to 7-membered heterocycloalkylene fused with one or more aromatic ring (s), (C3-C30) cycloalkylene with or without substituent(s), substituted or unsubstituted
(C3-C30)cycloalkylene fused with one or more aromatic ring(s), adamantylene with or without substituent(s),
(C7-C30)bicycloalkylene with or without substituent(s),
(C2-C30)alkenylene with or without substituent(s),
(C2-C30)alkynylene with or without substituent(s),
(C6-C30)ar(C1-C30)alkylene with or without substituent(s),
(C1-C30)alkylenethio with or without substituent(s),
(C1-C30)alkyleneoxy with or without substituent(s),
(C6-C30)aryleneoxy with or without substituent(s),
(C6-C30)arylenethio with or without substituent(s), —O— or —S—;
R₁, R₂, R₃₁ and Ar independently represent hydrogen, deuterium, halogen, (C1-C30)alkyl with or without substituent(s), (C6-C30) aryl with or without substituent(s), substituted or unsubstituted
(C6-C30)aryl fused with one or more (C3-C30)cycloalkyl(s) with or without substituent(s), (C3-C30)heteroaryl with or without substituent(s), 5- to 7-membered heterocycloalkyl with or without substituent(s), substituted or unsubstituted 5- to 7-membered heterocycloalkyl fused with one or more aromatic ring(s),
(C3-C30)cycloalkyl with or without substituent(s), substituted or unsubstituted (C3-C30)cycloalkyl fused with one or more aromatic ring(s), adamantyl with or without substituent(s),
(C7-C30) bicycloalkyl with or without substituent (s), cyano, NR₁₁R₁₂, BR₁₃R₁₄, PR₁₅R₁₆, P(═O)R₁₇R₁₈ [wherein R₁₁ through R₁₈ independently represent (C1-C30)alkyl with or without substituent(s),
(C6-C30)aryl with or without substituent(s) or (C3-C30)heteroaryl with or without substituent(s)], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s),
(C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s),
(C2-C30)alkynyl with or without substituent(s),
(C6-C30)aryloxycarbonyl with or without substituent(s),
(C1-C30)alkoxycarbonyloxy with or without substituent(s),
(C1-C30)alkylcarbonyloxy with or without substituent(s),
(C6-C30)arylcarbonyloxy with or without substituent(s),
(C6-C30)aryloxycarbonyloxy with or without substituent(s), carboxyl, bitro, 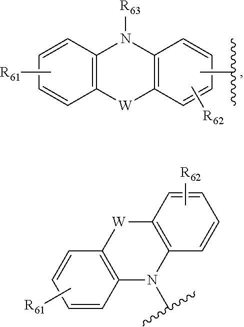
or hydroxyl, or each of them may be linked to an adjacent substituent via (C3-C30) alkylene or (C3-C30) alkenylene with or without a fused ring to form an alicyclic ring or a mono- or polycyclic aromatic ring;
W represents —(CR₅₁R₅₂)ₙ—, —(R₅₁) C═C(R₅₂)—, —N(R₅₃)—, —S—, —O—, —Si(R₅₄) (R₅₅)—, —P(R₅₆)—, —P(═O) (R₅₇)—, —C(═O)— or —B(R₅₈)—, and R₅₁ through R₅₈ and R₆₁ through R₆₃ are defined as for R₁ and R₂;
each of the heterocycloalkyl and heteroaryl contains one or more heteroatom(s) selected from B, N, O, S, P(═O), Si and P;
m represents an integer 1 or 2; and
n represents an integer 1 or 2.

2. The organic electroluminescent compound according to claim 1, which is selected from the following structures: 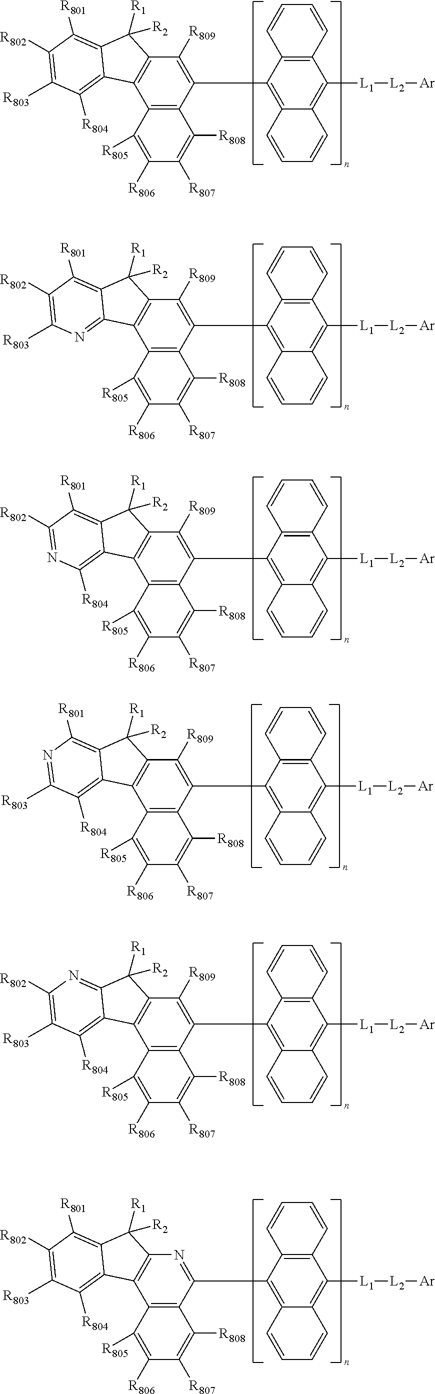 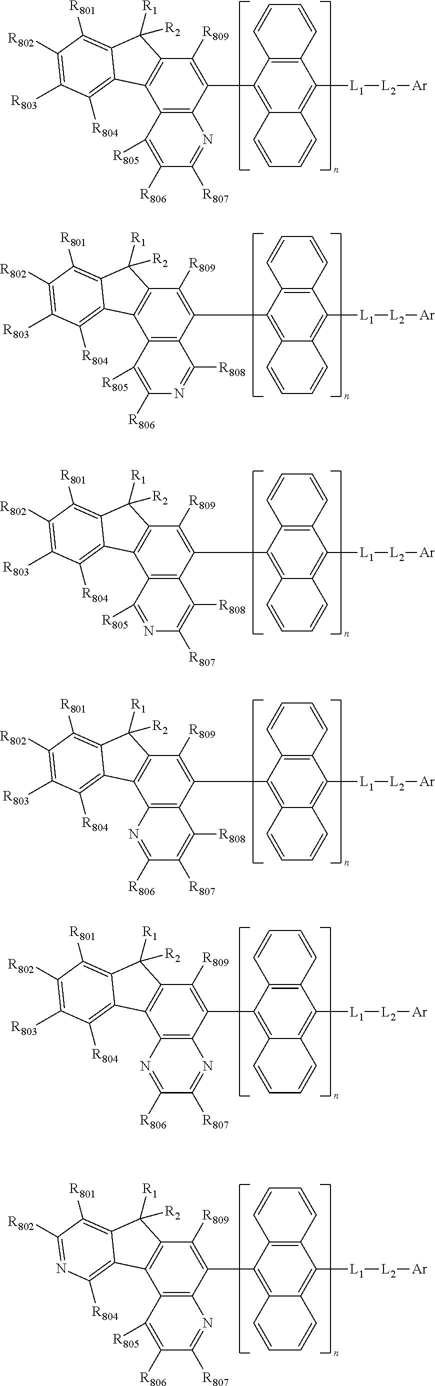
wherein,
L₁ and L₂, Ar and n are defined as in claim 1; and
R₈₀₁ through R₈₀₉ are defined as for R₁ and R₂ in claim 1.

3. The organic electroluminescent compound according to claim 1, wherein each substituent of L₁, L₂, R₁, R₂, R₁₁ through R₁₈, R₃₁, R₅₁ through R₅₈, R₆₁ through R₆₃ or Ar is independently substituted by one or more substituent(s) selected from a group consisting of hydrogen, deuterium, halogen, (C1-C30) alkyl with or without halogen substituent(s), (C6-C30)aryl, (C3-C30)heteroaryl with or without (C6-C30)aryl substituent(s), 5- to 7-membered heterocycloalkyl, 5- to 7-membered heterocycloalkyl fused with one or more aromatic ring(s), (C3-C30)cycloalkyl, (C3-C30)cycloalkyl fused with one or more aromatic ring(s), tri(C1-C30)alkylsilyl, di(C1-C30)alkyl(C6-C30)arylsilyl, tri(C6-C30)arylsilyl, adamantyl, (C7-C30)bicycloalkyl, (C2-C30)alkenyl, (C2-C30)alkynyl, cyano, carbazolyl, NR₂₁R₂₂, BR₂₃R₂₄, PR₂₅R₂₆, P(═O)R₂₇R₂₈ [wherein R₂₁ through R₂₈ independently represent (C1-C30)alkyl with or without substituent(s), (C6-C30)aryl with or without substituent(s) or (C3-C30)heteroaryl with or without substituent(s)], (C6-C30)ar(C1-C30)alkyl,
(C1-C30)alkyl(C6-C30)aryl, (C1-C30)alkyloxy, (C1-C30)alkylthio,
(C6-C30)aryloxy, (C6-C30)arylthio, (C1-C30)alkoxycarbonyl,
(C1-C30)alkylcarbonyl, (C6-C30)arylcarbonyl,
(C6-C30)aryloxycarbonyl, (C1-C30)alkoxycarbonyloxy,
(C1-C30)alkylcarbonyloxy, (C6-C30)arylcarbonyloxy,
(C6-C30)aryloxycarbonyloxy, carboxyl, nitro and hydroxyl, or is linked to adjacent substituent to form a ring.

4. An organic electroluminescent device comprising the organic electroluminescent compound according to any one of claims 1 to 3.

5. The organic electroluminescent device according to claim 4, which is comprised of a first electrode; a second electrode; and one or more organic layer (s) interposed between the first electrode and the second electrode, wherein the organic layer comprises one or more organic electroluminescent compound(s) according to claim 1 or 2 and one or more dopant(s) represented by Chemical Formula (2) or (3): 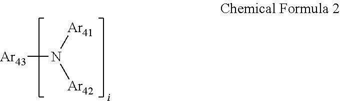
wherein,
Ar₄₁ and Ar₄₂ independently represent (C1-C30)alkyl with or without substituent(s), (C6-C30) aryl with or without substituent (s), (C4-C30) heteroaryl with or without substituent (s),
(C6-C30)arylamino with or without substituent(s),
(C1-C30)alkylamino, 5- to 7-membered heterocycloalkyl with or without substituent(s), substituted or unsubstituted 5- to 7-membered heterocycloalkyl fused with one or more aromatic ring (s),
(C3-C30)cycloalkyl with or without substituent(s), or substituted or unsubstituted (C3-C30) cycloalkyl fused with one or more aromatic ring (s), or Ar₄₂ and Ar₄₂ may be linked together via (C3-C30) alkylene or (C3-C30)alkenylene with or without a fused ring to form an alicyclic ring or a mono- or polycyclic aromatic ring;
when i is 1, Ar₄₃ represents (C6-C30)aryl with or without substituent(s), (C4-C30)heteroaryl with or without substituent(s) or a substituent selected from the following structures; 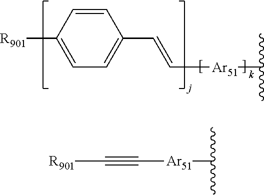
when i is 2, Ar₄₃ represents (C6-C60)arylene with or without substituent(s), (C4-C30)heteroarylene with or without substituent(s) or a substituent selected from the following structures; 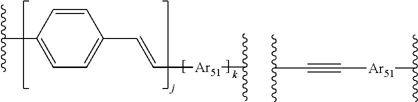
Ar₅₁ represents (C6-C60) arylene with or without substituent (s) or (C4-C30)heteroarylene with or without substituent(s);
R₉₀₁ independently represents hydrogen, deuterium,
(C1-C30)alkyl with or without substituent(s) or (C6-C30)aryl with or without substituent(s); each one of the heterocycloalkyl and heteroaryl contains one or more heteroatom(s) selected from B, N, O, S, P(═O), Si and P;
i represents an integer from 1 to 4;
j represents an integer from 1 to 4; and
k represents an integer 0 or 1: 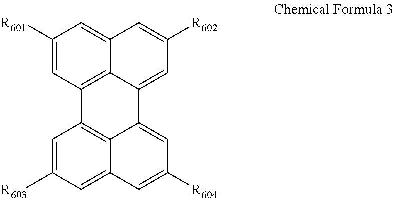
wherein,
R₆₀₁ through R₆₀₄ independently represent hydrogen, deuterium, halogen, (C1-C30)alkyl with or without substituent(s), (C6-C30) aryl with or without substituent(s), (C6-C30)heteroaryl with or without substituent(s), 5- to 7-membered heterocycloalkyl with or without substituent(s), substituted or unsubstituted 5- to 7-membered heterocycloalkyl fused with one or more aromatic ring (s),
(C3-C30)cycloalkyl with or without substituent(s), substituted or unsubstituted (C3-C30)cycloalkyl fused with one or more aromatic ring(s), adamantyl with or without substituent(s),
(C7-C30) bicycloalkyl with or without substituent (s), cyano, NR₄₁R₄₂, BR₄₃R₄₄, PR₄₅R₄₆, P(=0)R₄₇R₄₈ [wherein R₄₁ through R₄₈ independently represent (C1-C30)alkyl with or without substituent(s),
(C6-C30)aryl with or without substituent(s) or (C3-C30)heteroaryl with or without substituent(s)], tri(C1-C30)alkylsilyl with or without substituent(s), di(C1-C30)alkyl(C6-C30)arylsilyl with or without substituent(s), tri(C6-C30)arylsilyl with or without substituent(s), (C6-C30)ar(C1-C30)alkyl with or without substituent(s), (C1-C30)alkyloxy with or without substituent(s),
(C1-C30)alkylthio with or without substituent(s), (C6-C30)aryloxy with or without substituent(s), (C6-C30)arylthio with or without substituent(s), (C1-C30)alkoxycarbonyl with or without substituent(s), (C1-C30)alkylcarbonyl with or without substituent(s), (C6-C30)arylcarbonyl with or without substituent(s), (C2-C30)alkenyl with or without substituent(s),
(C2-C30)alkynyl with or without substituent(s),
(C6-C30)aryloxycarbonyl with or without substituent(s),
(C1-C30)alkoxycarbonyloxy with or without substituent(s),
(C1-C30)alkylcarbonyloxy with or without substituent(s),
(C6-C30)arylcarbonyloxy with or without substituent(s),
(C6-C30)aryloxycarbonyloxy with or without substituent(s), carboxyl, nitro or hydroxyl, or each of them may be linked to an adjacent carbon atom via (C3-C30)alkylene or (C3-C30)alkenylene with or without a fused ring to form a fused ring;
each one of the heterocycloalkyl and heteroaryl contains one or more heteroatom(s) selected from B, N, O, S, P(═O), Si and P.

6. The organic electroluminescent device according to claim 5, wherein the organic layer comprises one or more compound (s) selected from a group consisting of arylamine compounds and styrylarylamine compounds.

7. The organic electroluminescent device according to claim 5, wherein the organic layer further comprises one or more metal(s) selected from a group consisting of organic metals of Group 1, Group 2, 4th period and 5th period transition metals, lanthanide metals and d-transition elements in the Periodic Table of Elements, or complex(es) thereof.

8. The organic electroluminescent device according to claim 5, wherein the organic layer comprises an electroluminescent layer and a charge generating layer at the same time.

9. The organic electroluminescent device according to claim 5, which is a white light-emitting organic electroluminescent device wherein the organic layer simultaneously comprises one or more organic electroluminescent layer(s) emitting blue, red or green light.
